(19) United States
(12) Patent Application Publication
(10) Pub. No.: US 2023/0333087 A1
(43) Pub. Date: Oct. 19, 2023

(54) METHODS FOR MONITORING PATIENT RESPONSE TO TREATMENT OF RETINAL OXIDATIVE DISEASES

(71) Applicant: BioJiva LLC, Los Altos, CA (US)

(72) Inventors: Karsten SCHMIDT, Los Altos, CA (US); Mikhail S. SHCHEPINOV, Oxford (GB)

(21) Appl. No.: 18/167,781

(22) Filed: Feb. 10, 2023

Related U.S. Application Data
(60) Provisional application No. 63/309,468, filed on Feb. 11, 2022.

(51) Int. Cl. G01N 33/50

(52) U.S. Cl. CPC G01N 33/5008

(57) ABSTRACT

Disclosed are methods for assessing the presence or absence of a therapeutic concentration of a deuterated docosahexaenoic acid during treatment of a patient with a retinal oxidative disease.

### CROSS-REFERENCE

This application claims the benefit of U.S. Provisional Pat. Application No. 63/309,468, filed Feb. 11, 2022, and 63/309,471, filed Feb. 11, 2022, which are incorporated by reference herein in their entirety.

### BACKGROUND

A number of retinal diseases are mediated, at least in part, by lipid peroxidation of arachidonic acid or docosahexaenoic acid (DHA) found in the peripherical rods and cones of the retina. Such retinal diseases include, but not limited to, wet and dry age-related macular degeneration (including geographic atrophy associated therewith), retinitis pigmentosa, diabetic retinopathy, cataracts, and Stargardt Disease.

The use of deuterated polyunsaturated fatty acids or esters thereof, including deuterated docosahexaenoic acid (D-DHA) or an ester thereof, to treat these diseases as disclosed in U.S. Pat. No. 10,058,522 which is incorporated herein by reference in its entirety. Specifically, the underlying pathology of these diseases includes the lipid peroxidation at the bis-allylic positions of the polyunsaturated fatty acids (PUFA) in the peripheral or outer portions of the rods and cones found in the retina. These rods and cones comprise a significant amount of DHA making up between 30% to 60% of the fatty acids in these outer segments.

Treating oxidative retinal diseases with D-DHA or an ester thereof is complicated by the fact that it can take weeks to months after the initiation of treatment to reach a therapeutic concentration in the retina. Moreover, since the rods and cones are inaccessible in living subjects, monitoring the progress of a patient in reaching a therapeutic concentration of the drug in the retina is not feasible. One can only indirectly assess this by periodically monitoring the retina for the progression of the disease with the assumption that any apparent abatement in disease progression is attributable to the treatment. However, such an approach fails to address whether the failure to see such abatement is attributable to the lack of efficacy of the drug or inadequate therapeutic levels of the of the drug in the target tissue(s), whereby an adequate D-DHA substitution level is the key figure of merit for drug efficacy, i.e. the proportion of D-DHA relative to total DHA has to reach a therapeutically effective percentage of the total DHA pool absorbed upon daily dosing.

As to the latter, the uptake of D-DHA is controlled by the total amount of DHA consumed by the patient. Stated differently, the more naturally occurring DHA consumed during therapy, the more it dilutes the relative percentage of administered D-DHA absorbed with the total DHA pool. Still further, an individual’s diet rich in seafood or in fish oil (such as found in certain medicaments) can reduce the relative D-DHA uptake of the drug into the body. Accordingly, each patient, being treated with a fixed dose of the drug, will absorb different relative amounts of the drug and those amounts will vary from day to day and from patient to patient. This raises the conundrum of how a clinician can ascertain if the patient is progressing in a suitable manner to achieving a therapeutic concentration of the drug in the rods and cones in an expedient manner. This is particularly important because the longer it takes to achieve a therapeutic concentration the greater the risk of retinal damage and loss of additional vision.

As is apparent, methods that allow the clinician to monitor patients to determine whether they are properly progressing to a therapeutic concentration of the drug in the retina is an unmet and critical need.

### SUMMARY

Disclosed are methods that allow for a clinician to confirm that the retinal uptake of D-DHA by a patient is progressing properly by confirming that a certain steady state concentration of this drug is confirmed in the patient’s plasma and/or red blood cells. The absorption and tissue distribution of D-DHA follows a first order kinetics, and as shown in the examples below, the steady state concentration of D-DHA in plasma occurs after about 21 to 28 days after initiation of therapy. Alternatively, the steady state concentration of D-DHA in red blood cells occurs after about 33 to 44 days after initiation of therapy. When such a steady state is achieved, it evidences that patients have a maximal concentration of the drug in their blood and the maximum D-DHA substitution levels relative to total DHA have been reached. As the blood serves to a depot that delivers the drug to the retina, the clinician can confirm that a steady concentration in the blood correlates with proper uptake by the patient and maximal delivery of the drug to the retina. On the other hand, the failure to achieve a steady state concentration in blood in a timely fashion evidences that the patient either is consuming foods or medicaments rich in DHA. In either case, the patient may be required to adjust their diet and/or the patient’s dosing of the drug might need to be increased.

As also shown in the examples below, the plasma steady state concentration of D-DHA in the plasma precedes that in the retina by approximately 7 to 10 weeks whereas the steady state concentration in red blood cells precedes that in the retina by approximately 5 to 7 weeks. Moreover, for instance, based on a mean daily dietary intake of about 130 mg of DHA per day (which represents the 90th percentile of the mean usual DHA intake by males >51 years of age in the US as an example), at a dosing regimen of 250 mg/day, the steady state concentration of the drug in the plasma, red blood cells, and in the retina is about 65% of the total amount of DHA present including D-DHA. At a dosing regimen of 500 mg/day, the steady state concentration of the drug in the plasma and in the retina is about 80%, and at a dosing regimen of 1,000 mg/day, the steady state concentration of the drug in the plasma and in the retina is about 88%. Thus, while the time to reach the steady state relative D-DHA concentration at these three dosing levels remains the same, there is a significant increase in the relative concentration of the drug at steady state using a higher dose.

Based on the above, the dosing of D-DHA or ester thereof typically ranges from about 150 mg/day to about 1,000 mg/day and preferably from about 250 mg/day to about 500 mg/day. In one preferred embodiment, the dose employed is sufficient to achieve a steady state concentration of D-DHA of about 50% or more based on the total amount of DHA present including D-DHA.

Based on the finding that the steady state concentration of D-DHA in either plasma or red blood cells correlates well with the steady-state concentration in the retina, one can generate standardized concentration curves for each dose of this drug based on either the plasma or red blood cells. Such standardized curves will correlate with the concentration of the drug using different doses and measured at various times from the start of therapy to the concentration where a steady state should be reached in plasma or red blood cells. Such standardized curves can then be used to monitor and evaluate the overall response to treatment for a given patient.

Accordingly, in one embodiment there is provided a method for monitoring a patient for uptake of D-DHA wherein said method comprises:
periodically administering to said patient an effective dose of D-DHA or an ester thereof;
obtaining one or more blood samples from said patient after the start of therapy;
assessing the amount of D-DHA in said sample relative to the total amount of DHA;
comparing the assessed amount of D-DHA against a standard concentration curve wherein said curve is based on a specific dose of D-DHA or ester thereof employed, the blood component being assessed, and the said length of time from start of therapy; and
determining if the patient is achieving proper D-DHA substitution levels based on said curve.

In one embodiment, the blood component being assessed is plasma.

In one embodiment, the blood component being assessed is red blood cells.

In one embodiment, the length of time between start of therapy and testing is from about 7 to about 45 days. In one embodiment, the length of time between start of therapy and testing is at least about 14 days. In another embodiment, the length of time between start of therapy and testing is at least about 30 days.

In one embodiment, there is provided a method for monitoring a patient for uptake of D-DHA wherein said method comprises:
periodically administering to said patient an effective dose of D-DHA or an ester thereof wherein said does is about 250 mg/day;
obtaining one or more plasma samples from said patient after the start of therapy;
assessing the amount of D-DHA in said sample relative to the total amount of DHA;
comparing the assessed amount of D-DHA against a standard concentration curve wherein said curve is based on the said length of time from start of therapy; and
determining if the patient is properly absorbing D-DHA based on said curve.

In one embodiment, there is provided a method for monitoring a patient for uptake of D-DHA wherein said method comprises:
periodically administering to said patient an effective dose of D-DHA or an ester thereof wherein said does is about 500 mg/day;
obtaining one or more red blood cell samples from said patient after the start of therapy;
assessing the amount of D-DHA in said sample relative to the total amount of DHA;
comparing the assessed amount of D-DHA acid against a standard concentration curve wherein said curve is based on the said length of time from start of therapy; and
determining if the patient is properly absorbing D-DHA based on said curve.

In one embodiment, there is provided a method for monitoring a patient for uptake of D-DHA wherein said method comprises:
periodically administering to said patient an effective dose of D-DHA or an ester thereof wherein said does is about 1,000 mg/day;
obtaining one or more red blood cell samples from said patient after the start of therapy;
assessing the amount of D-DHA in said sample relative to the total amount of DHA;
comparing the assessed amount of D-DHA acid against a standard concentration curve wherein said curve is based on the said length of time from start of therapy; and
determining if the patient is properly absorbing D-DHA based on said curve.

In one embodiment, when the concentration of deuterated docoshexaenoic acid is less than that provided by the standardized curve, the clinician has the option of either prescribing a modification to the patient’s diet to reduce the amount of naturally occurring DHA consumed per day and/or to increase the amount of drug administered.

In one embodiment, the method includes comparing the amount of deuterated docosahexaenoic acid in a sample to a minimum therapeutic concentration of at least 50% of deuterated docosahexaenoic acid based on the total amount of docosahexaenoic acid, including deuterated docosahexaenoic acid, in the sample to determine if the patient has a therapeutic concentration or a sub-therapeutic concentration of deuterated docosahexaenoic acid.

In one embodiment, the therapeutic concentration of deuterated docosahexaenoic acid is set at 60%, 70%, or even 80% as a therapeutic target for a given patient.

In one embodiment, the method includes increasing the dose the dose of deuterated DHA administered to the patient if the amount of deuterated docosahexaenoic acid in the sample is less than the minimum therapeutic concentration.

In one embodiment, the method includes restricting the patient’s consumption of dietary docosahexaenoic acid during therapy with deuterated docosahexaenoic acid.

In one embodiment, the method includes restricting the patient’s consumption of dietary docosahexaenoic acid (i.e., docahexanoic acid consumed by the patient, not including the amount of deuterated docosahexaenoic acid administered) to no more than about 132 mg per day.

### BRIEF DESCRIPTION OF THE DRAWINGS

FIG. 1 illustrates standardized curves showing the increase in concentration of D-DHA using three different markers (plasma, red blood cells and the retina) in a cohort of mice on a customized rodent diet containing 0.5% w/w D-DHA at four different time points (8, 19, 38, and 78 days after first D-DHA exposure).

FIGS. 2, 3 and 4 illustrate standardized curves showing the increase in concentration of D-DHA for three different dosing regimens and using different markers (plasma, red blood cells and the retina) over a time course from start of therapy to reaching steady state in patients with a mean dietary intake of about 130 mg DHA per day.

FIGS. 5A-5E collectively show a chemical diagram of the steps in iron catalyzed lipid peroxidation of phospholipids containing DHA and the formation of CEP. FIG. 5A shows that iron catalyzes hydroxyl radical generation through the Fenton reaction and Haber-Weiss reaction. FIG. 5B shows ROS driven hydrogen abstraction off bis-allylic sites generates free radicals, which rapidly react with oxygen to form lipid peroxyl radicals. FIG. 5C shows that newly formed ROS species then abstract bis-allylic hydrogen atoms from the neighboring PUFAs, thus sustaining the LPO chain reaction cycle. FIG. 5D shows D-DHA was used in the example. FIG. 5E shows DHA peroxidation generates multiple oxidation products including reactive carbonyls such as HHE and HOHA, which can give rise to protein modifications, including OBA, CEP and MDA adducts. The substitution of deuterium for hydrogen atoms inhibits the rate-limiting step of ROS-driven abstraction off bis-allylic sites.

FIGS. 6A-6G show charts and images demonstrating D-DHA protection against iron induced retinal autofluorescence and degeneration. Mice were fed with D-DHA for 77 days, followed by a switch to DHA for 73 days for a total of 150 days of feeding. FIG. 6A shows %D-DHA in neural retina and RPE-choroid. FIG. 6B shows a timeline of mice being fed with either D-DHA or DHA for 1 week, 2 week, or 4 weeks beginning at 2 months age, then given an intravitreal injection of iron in one eye and control normal saline in the other. Mice were continued on their respective diets until their final evaluation. FIG. 6C shows retinal AF area in BAF cSLO images from mice fed with 4 weeks of DHA or D-DHA at 1 week after iron injection (designated 4+1 wk). The cSLO and OCT imaging was performed at 1 week after IVT iron versus saline injection. FIGS. 6D-6G show representative BAF cSLO images in mice fed D-DHA or DHA for 1 week, given IVT injections, then euthanized a week later (1+1 wk), or fed D-DHA for two weeks, given IVT injections, then euthanized a week later (2+1 wk), etc (d and e), IRAF cSLO images (f), horizontal OCT b scans (g) are shown. Abbreviations used for the figures: SLO, scanning laser ophthalmoscopy; OCT, optical coherence tomography; BAF, blue autofluorescence ; IRAF, infrared autofluorescene ; ONL, outer nuclear layer. White rrows indicate hyper-AF spots induced by iron. White broken arrows indicate vesicles in damaged RPE cells. White ines indicate the position and orientation of horizontal OCT b scans in panel e. white stars indicate the vortex vein that was used as a landmark for the corresponding position of the OCT scan in IRAF SLO images. N=3 mice/group in c; N=10 mice/group in b, and d-f. Error bars indicate mean ± SEM. (** P < 0.01).

FIGS. 7A-7F show that D-DHA protected retinal function and structure against iron injection. FIG. 7A show graphs showing electroretinography amplitudes 4 weeks after dietary dosing of either D-DHA or DHA. FIG. 7B shows electroretinography amplitudes reconducted at 1 week after an intravitreal injection of iron or saline. FIGS. 7C and 7D shows images of toluidine blue staining conducted on plastic sections prepared at 1 week after injections. The enlarged image is from section from mouse fed with DHA diet for 4 weeks then given IVT iron and euthanized a week later. Black dashed arrow indicates atrophic RPEs; white dashed arrows indicate vesicles in damaged RPE cells; Black solid arrows indicate infiltrated myeloid cells. Two- sample t-tests were performed to compare the total retinal thickness and outer retinal thickness between DHA-Fe group and D-DHA-Fe group at each different location. FIGS. 7E and 7F show spider graphs of the mean thickness of each retinal layer. Error bars indicate mean ± SEM of total retinal thicknesses and outer retina thickness (ONL to RPE) in the ventral (inferior) - dorsal (superior) axis at the positions indicated on the x-axis. All statistical comparisons were made using SAS v9.4 (SAS Institute Inc., Cary, NC). No correction for multiple comparisons was performed due to the exploratory nature of this small study. Error bars indicate mean ± SEM. * P < 0.05. Scale bar: 50 µM. N=8-10/group for electroretinography. N=3/group for retina thickness measures.

FIGS. 8A-8G shows that D-DHA prevented the formation of CEP, an immunogenic protein adduct, uniquely derived from DHA oxidation. FIG. 8A shows epifluorescence photomicrographs of co-labelling for carboxyethyl pyrrole (CEP-red) and rhodopsin (green) on cryosections from mice fed with 4 weeks of D- or DHA at 4 h after intravitreal injection of iron or saline. FIG. 8B shows immunolabeling for CEP at 1 week after injections. FIG. 8C shows an enlarged image of co-labelling for CEP and rhodopsin corresponding to FIG. 8B. FIG. 8D shows an enlarged image of immunolabelling for CEP corresponding to FIG. 8B. FIG. 8E shows immunolabelling for L-Ft at 1 week after injections. FIGS. 8F and 8G show chart of quantification of pixel density of immunolabeling for CEP and L-Ft. White arrows indicate immunolabeling for CEP. Abbreviations used in the figures: CEP, carboxyethyl pyrrole; INL, inner nuclear layer; ONL, outer nuclear layer; RPE, retinal pigmented epithelium. Representative images are shown from N=4 mice/group. Scale bar: 50 µm. Error bars indicate mean ± SEM. ** P < 0.01, **** P <0.0001).

FIG. 9 shows qPCR indicating that D-DHA protected against iron induced oxidative stress, inflammation, and retinal cell death. Relative mRNA levels in the neural retina of the indicated genes from mice fed with 4 weeks of D-DHA or DHA at 1 week after iron or saline injection. Error bars indicate mean ± SEM. N=3-4 mice/group. (* P < 0.05, ** P < 0.01, **** P <0.0001, **** P <0.0001).

FIGS. 10A-10E shows that D-DHA prevented iron induced acute RPE atrophy and progressive geographic atrophy development. Mice were fed with 4 weeks of D-DHA or DHA before receiving intravitreal injection of iron in one eye and control normal saline in the other. cSLO and OCT images were acquired at 4 weeks after iron or saline injection. FIGS. 10A-10C shows representative BAF cSLO images (a), IRAF cSLO images (b), and OCT scans (c). White lines indicate the positions of horizontal OCT scans. Black arrows indicate hyper-AF and hypo-AF lesions in IRAF cSLO images, corresponding to atrophic RPEs in OCT scans. White arrows indicate ONL thinning in OCT scans. FIGS. 10D-10E shows toluidine blue staining conducted on plastic sections prepared at 4 weeks after injections. Black arrows indicate atrophic RPEs and vesicles within RPEs. White arrows indicate hypertrophic RPEs. Representative images are shown from N=4 mice/group. Scale bar: 50 µm.

FIGS. 11A-11C shows LC/MS analysis of lipids extracted from D-DHA-containing diet. Free fatty acid mixtures resulting from sample extraction and saponification were dissolved in ethanol and injected in 5 µl volumes onto an Agilent XDB-C18 liquid chromatography column (1 mm x 150 mm) through which running solvents were pumped at 100 µ1/min. Solvent A was 70% CH₃CN (v/v) and 0.1% formic acid (m/v). Solvent B was 0.1% formic acid (m/v) in neat CH₃CN. The initial composition of the running solvent was 70% B for 5 min, increasing to 100% B between 5 and 30 min. DHA eluted at 24.7 min. The column effluent was alkalinized with 150 mM NH₄OH before ESI-MS analysis on a 4000 QTrap (Sciex) operating in enhanced negative mode over an m/z range of 320 --- 345 and a scan rate of 250 /sec. These procedures verified that laboratory rodent diet contained DHA but no detectable D-DHA, while the experimental D-DHA supplemented diet contained only trace amounts of ordinary DHA (a). A ¹³C correction applied to the DHA signals verified that the peak at m/z 327.2 represented 78.4 % of the DHA and ¹³C-containing isotopologues. Peaks corresponding to DHA with 8, 9, 10, 11, 12, and 13 deuterium substitutions were readily identified in the experimental diet, and in samples of neural retina and RPE. The relative distribution of DHA isotopologues in neural retina and RPE samples was indistinguishable from the relative distribution in the experimental D-DHA supplemented diet. After ¹³C corrections were applied to the integrated peaks, it was determined that the area of the peak centered at 337.2 (corresponding to D₁₀-DHA) comprised 45.6 % of the area of all deuterium-containing DHA isotopologue peaks. Fatty acids extracted from the neural retina and RPE eluted as 3 peaks (b). The TIC shown was derived from enhanced negative mode scans from m/z 320-345 at 250 m/z/min. Mass spectra for the three labeled peaks (c). Peak 1 shows DHA at 327.2, a ¹³C-containing isotopologue at 328.2, and isotopologues containing 8, 9, 10, 11, and 12 deuterium substitutions at corresponding m/z values. Relative peak areas were indistinguishable from the relative peak areas observed in the experimental diet. Peak 2 shows some DHA (a tail from peak 1), 329.2/330.2 peaks indicating DPA, and a set of peaks suggesting that they represented D₈-DPA, D₉-DPA, D₁₀-DPA, D₁₁-DPA, and D₁₂-DPA. Because these species were not present in the chow, they appear to represent D-DHA species that have been reduced to D-DPA species. The relative peak areas of D₈-DPA and D₉-DPA were slightly greater than the relative peak areas of Ds-DHA and D₉-DHA, possibly reflecting greater likelihood of reduction to the corresponding DPA species when the degree of deuterium substitution is lower. Peak 3 eluting at 27.9 min most likely represents docosatetraenoic acid (DTA) of the n-6 series, and no deuterium-substituted isotopologues were observed.

FIGS. 12A-12D show that D-DHA showed a dose-dependent protection effect against iron induced retinal AF. Beginning at 2 months age, mice were fed with D-DHA or DHA for 1 week, 2 weeks, 3 weeks, and 4 weeks before receiving an intravitreal injection of iron in one eye and control normal saline in the other. At 1 week after iron versus saline injection, BAF cSLO images were acquired from multiple mice fed with D-DHA or DHA for 1 week (1+1wk) (a), 2 weeks (2+1wk) (b), 3 weeks (3+1wk) (c), and 4 weeks (4+1wk) (d) prior to injections. BAF cSLO pairs of images from the same mouse were presented in the same row, images from different mice with the same treatment (iron or saline) were presented in the same column.

FIGS. 13A-13B show the long-term protective effect of D-DHA against chronic geography atrophy development. Mice were fed with D-DHA or DHA for 4 weeks before receiving intravitreal injection of iron in one eye and control normal saline in the other. BAF cSLO images (a) and OCT scans (b) were acquired at 4 weeks after injections. Pairs of BAF/IRAF images from the same mouse were presented in the same row, images from different mice with the same treatment (iron or 781 saline) were presented in the same column.

FIG. 14 shows dose-response of D-DHA protection against iron induced retinal damage. Representative cSLO BAF (a) and OCT images (b) from animals fed with control DHA diet (left column) and D-DHA diets for increasing periods of time before intravitreal injection of FAC. Images were acquired one week after iron injection. Retinal D-DHA levels at 4+1 weeks as measured (see Table 1); levels at other time points were extrapolated assuming a 1^{st} order uptake kinetics as shown in FIG. 2. Appearance of autofluorescent spots served as quantitative measure of damage or protection by D-DHA (a). OCT scans show retinal thinning and destruction of the photoreceptor + RPE layers with no or low levels of retinal D-DHA and increasing preservation with higher D-DHA concentrations (b). Protection effect refers to the reduction (%) of AF area quantified by ImageJ software, N=3-5/group.

### DETAILED DESCRIPTION

Disclosed are methods to monitor a patient’s response to the treatment of retinal diseases, mediated at least in part, by lipid peroxidation.

Disclosed are methods for monitoring the uptake of D-DHA in patients being treated for oxidative retinal diseases. Before describing the invention in more detail, the following terms are defined. Terms that are not defined are given their definition in context or are given their medically acceptable definition.

The terminology used herein is for the purpose of describing particular embodiments only and is not intended to be limiting of the invention. As used herein, the singular forms “a”, “an” and “the” are intended to include the plural forms as well, unless the context clearly indicates otherwise.

As used herein, the term “optional” or “optionally” means that the subsequently described event or circumstance can or cannot occur, and that the description includes instances where the event or circumstance occurs and instances where it does not.

As used herein, the term “about” when used before a numerical designation, e.g., temperature, time, amount, concentration, and such other, including a range, indicates approximations which may vary by ( + ) or ( - ) 15%, 10%, 5%, 1%, or any subrange or subvalue there between. Preferably, the term “about” when used with regard to a dose amount means that the dose may vary by +/- 10%.

As used herein, the term “comprising” or “comprises” is intended to mean that the compositions and methods include the recited elements, but not excluding others.

As used herein, the term “consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention.

As used herein, the term “consisting of” shall mean excluding more than trace elements of other ingredients and substantial method steps. Embodiments defined by each of these transition terms are within the scope of this invention.

As used herein and unless the context dictates otherwise, the term “an ester thereof” refers to a C₁-C₁₀ alkyl esters, glycerol esters (as defined herein and including monoglycerides, diglycerides and triglycerides), sucrose esters, phosphate esters, and the like. The particular ester group employed is not critical provided that the ester is pharmaceutically acceptable (non-toxic and biocompatible). In one embodiment, the ester is a C₁-C₆ alkyl ester that is preferably an ethyl ester.

As used herein, the terms “deuterated DHA”, “D-DHA” or “deuterated docosahexaenoic acid or ester thereof” refers to a docosahexaenoic acid or an ester thereof having deuteration as described below. Prior to describing said deuteration, the structure of docosahexaenoic acid and specific sites therein are provided in formula A below:

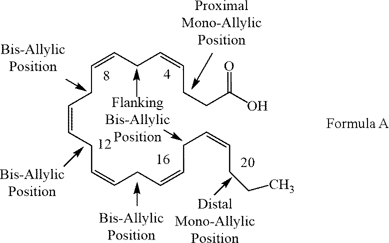

As to deuteration, such is described as an average based on a population of such DHA compounds comprising a total deuteration of from about 92 to about 96 percent at the bis-allylic sites wherein said total deuteration is present as follows:
a) from about 87 percent to about 92 percent CD₂ moieties at the bis-allylic sites;
b) from more than about 6 about to 12 percent CHD moieties at the bis-allyic sites; and
c) about 2 percent or less of CH₂ moieties at the bis-allylic sites,
provided that the aggregate number of hydrogen and deuterium at the bis-allylic positions equals 10.

In one embodiment, the population of deuterated DHA has total deuteration of from about 93 to about 96 percent at the bis-allylic sites wherein said total deuteration is present as follows:
a) from about 87 to about 92 percent CD₂ moieties at the bis-allylic sites;
b) from about 6.5 to about 12 percent CHD moieties at the bis-allylic sites; and
c) about 1.5 percent or less of CH₂ moieties at the bis-allylic sites,
provided that the aggregated number of deuterium and hydrogen atoms at the bis-allylic positions equals 10.

In one embodiment, the population of deuterated DHA is characterized as having a total deuteration of from about 92 to about 95 percent at the bis-allylic sites wherein said total deuteration is present as follows:
a) about 88 to 92 percent CD₂ moieties at the bis-allylic sites;
b) about 6.5 to 12 percent CHD moieties at the bis-allyic sites;
c) about 1.5 percent or less of CH₂ moieties at the bis-allylic sites; and
d) on average no more than an aggregate of 25% total deuteration at both of the mono-allylic sites
provided that the total number of hydrogen and deuterium at the bis-allylic positions equals 10.

The extent of deuteration at the two mono-allylic differs due to the steric hindrance imparted by the carboxyl or carboxyl ester. In one embodiment, the extent of deuteration at the proximal mono-allylic site is from about 0.5% to about 5%. In another embodiment, the extent of deuteration at the proximal mono-allylic site is from about 1% to about 5%. Stated differently, on average, only about 0.5% to about 5% or 1% to about 5% of the hydrogen atoms found at the proximal mono-allylic site of a composition comprising a population of deuterated DHA have been replaced by deuterium.

In one embodiment, the level of deuteration at the distal mono-allylic site is from about 10% to about 20%. In another embodiment, the level of deuteration at he distal mono-allylic sites is from about 12% to about 18%. Stated differently, on average, only about from 10% to about 20% or from 12% to about 18% of the hydrogen atoms found at the distal mono-allylic site of a composition comprising a plurality of deuterated DHA have been replaced by deuterium.

In one embodiment, the deuterated docosahexanoic acid or ester thereof comprises a population of a compound of formula I:

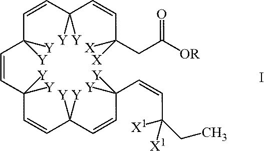

where R is hydrogen or C₁-C₁₀ alkyl;
each X is independently hydrogen or deuterium wherein the aggregate amount of the amount of deuterium defined by both X groups is such that, on average, the total amount of deuteration at the carbon atom is less than about 5%;
each X¹ is independently hydrogen or deuterium wherein the aggregate amount of the amount of deuterium defined by both X¹ is such that, on average, less than about 25% of the X¹ groups are deuterium and the remainder are hydrogen;
each Yis independently hydrogen or deuterium wherein the specific value for each Y is selected such that on average:
   a) from about 87 to about 92 percent of the Y groups on each carbon atom are deuterium;
   b) from more than about 5 about to 12 percent of the Y groups on each carbon atom are substituted with a single hydrogen and a single deuterium; and
   c) less than about 2 percent of the Y groups of each carbon atom are substituted with two hydrogen atoms;
   provided that the sum of all Y groups equal 10.

In one embodiment, the composition comprising the compound of formula I comprises less than 1.5 percent of the carbon atoms at the bis-allylic sites being substituted with two hydrogen atoms.

In one embodiment, the compositions described herein do not replace hydrogen with deuterium other than at the mono-allylic and bis-allylic sites. As such, the level of deuterium found in the remaining sites in DHA is at its natural abundance.

In one embodiment, the deuterated DHA provide a pharmaceutical composition comprising a pharmaceutically acceptable carrier and an effective amount of deuterated DHA as described herein.

The populations described above are useful in treating retinal diseases mediated, at least in part, by lipid peroxidation of DHA found in the outer rods and cones of the retina. Such methods include administering to a patient in need thereof a composition comprising a population of deuterated DHA as described herein. In one embodiment, the composition comprising a population of deuterated DHA is administered in a pharmaceutically acceptable formulation.

When describing the population ex vivo, the terms “D-DHA or drug” refer to deuterated docosahexaenoic acid or an ester thereof. When describing the population in vivo, the ester is hydrolyzed in the gastro-intestinal tract and, in the environment of the retina, docosahexaenoic acid is incorporated into a glycerol ester such as a phospholipid, including cardiolipin, plasmalogen and those of the formula II:

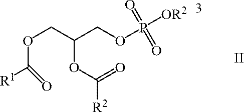

where R¹ is a fatty acid residue or the residue of docosahexaenoic acid, R² is the residue of docosahexaenoic acid, and R³ is choline, ethanolamine, serine, inositol or hydrogen, a mono-or divalent salt. Unlike fatty acids found elsewhere in the body, the retina can comprise residues of deuterated docosahexaenoic acids at both R¹ and R². Accordingly, this invention provides for phospholipids of formula II where R¹ is selected from a residue of a saturated fatty acid or the residue of docosahexaenoic acid and R² is the residue of docosahexaenoic acid. As to the terms “residue of a fatty acid” or “residue of docosahexaenoic acid”, each of these refers to the ester bond formed between a carboxyl group and a hydroxyl group of glycerol coupled with the elimination of water.

In one embodiment, deuteration at other sites of docosahexaenoic acid or an ester thereof is unaffected and, hence, the level of deuteration at sites other than the bis-allylic and mono-allylic sites is at the natural abundance.

The term “naturally occurring docosahexaenoic acid” refers to any and all sources of DHA where the abundance of deuterium is based on its natural abundance.

As used herein, the term “phospholipid” refers to any and all phospholipids that are components of the cell membrane. Included within this term are phosphatidylcholine, phosphatidylethanolamine, phosphatidylserine, and sphingomyelin. In the motor neurons, the cell membrane is enriched in phospholipids comprising arachidonic acid.

The term “bis-allylic site” refers to the methylene group (CH₂) separating two double bonds.

The term mono-allylic site” refers to the methylene group have an adjacent neighboring double bond on one side and a further methylene group on the opposite side.

The term “retinal diseases” refers to any and all retinal diseases mediated, at least in part, by reactive oxygen species (ROS). Such include, by way of example only, Wet or Dry Age-related macular degeneration (AMD), Retinitis Pigmentosa (RP), Stargardt Disease (SD), Diabetic Retinopathy (DR), cataracts, and the like.

The term “oxidized PUFA products” refer to any oxidized form of a polyunsaturated fatty acid as well as any and all metabolites formed from the oxidized PUFA including reactive aldehydes, ketones, alcohols, carboxyl derivatives which are toxic to the cell where found in a phospholipid, a lipid bilayer, or as an enzyme substrate.

As used herein, the term “pathology of a disease” refers to the cause, development, structural/functional changes, and natural history associated with that disease. Included in the pathology of the disease is the reduction in cellular functionality.

The term “therapeutic concentration” means a concentration of a deuterated DHA that reduces the rate of an oxidative retinal disease. Such a concentration is predicated on replacing at least about 20 percent of the DHA in the outer segments of the retina’s rods and cones with deuterated DHA as described herein and preferably at least about 50 percent, preferably at least about 60 percent, more preferably at least about 70%, and mostpreferably at least about 80 percent. To achieve this level of replacement level, dosing of DHA over a period of time (weeks to several months) is necessary as deuterated DHA is slowly exchanged in the rods of cones as well as the limited uptake. In general, about 0.1 to 1 gram of deuterated DHA is administered daily. Preferably, the administration of deuterated DHA is either 250 mg/day or 500 mg/day. The deuterated DHA is delivered in a pharmaceutically acceptable manner preferably (optionally?) including the use of a pharmaceutically acceptable excipient. Over a period of at least 2 weeks or 4 weeks, sufficient deuterated DHA is incorporated into the rods and cones to provide for therapy.

As used herein, the term “patient” refers to a human patient or a cohort of human patients suffering from a neurodegenerative disease treatable by administration of deuterated DHA. The term “subject” refers to a mammalian subject.

As used herein, the term “maintenance dose” refers to a dose of deuterated DHA that is less than the initial dose and is sufficient to maintain a therapeutic concentration of deuterated DHA in the outer rods and cones of the retina cells. In one embodiment, the maintenance dose deuterated DHA is about 30 to about 70% of the initial dose of deuterated DHA. It is understood that the initial dose is intended to increase the concentration of deuterated DHA in the outer rods and cones of the retina until a therapeutic concentration is achieved. At that point and at the discretion of the attending clinician, backing down the dose of deuterated DH may be advantageous such that the maintenance dose is sufficient to maintain the therapeutic concentration without further increases in the intra-retinal concentration.

As used herein, the term “periodic dosing” refers to a dosing schedule that substantially comports to the dosing described herein. Stated differently, periodic dosing includes a patient who is compliant at least 75 percent of the time over a 30-day period and preferably at least 80% compliant with the dosing regimen described herein. In embodiments, the dosing schedule contains a designed pause in dosing. For example, a dosing schedule that provides dosing 6 days a week is one form of periodic dosing. Another example is allowing the patient to pause administration for from about 3 or 7 or more days (e.g., due to personal reasons) provided that the patient is otherwise at least 75 percent compliant. Also, for patients who transition from the loading dose to the maintenance dose, compliance is ascertained by both the loading dose and the maintenance dose.

As used herein, the term “pharmaceutically acceptable salts” of compounds disclosed herein are within the scope of the methods described herein and include acid or base addition salts which retain the desired pharmacological activity and is not biologically undesirable (e.g., the salt is not unduly toxic, allergenic, or irritating, and is bioavailable). When the compound has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g., alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound has an acidic group, such as for example, a carboxylic acid group, it can form salts with metals, such as alkali and earth alkali metals (e.g., Na⁺, Li⁺, K⁺, Ca²⁺, Mg²⁺, Zn²⁺), ammonia or organic amines (e.g., dicyclohexylamine, trimethylamine, trimethylamine, pyridine, picoline, ethanolamine, diethanolamine, triethanolamine) or basic amino acids (e.g., arginine, lysine, and ornithine). Such salts can be prepared in situ during isolation and purification of the compounds or by separately reacting the purified compound in its free base or free acid form with a suitable acid or base, respectively, and isolating the salt thus formed.

### Compound Synthesis

Deueterated DHA compositions as described herein are obtained in one synthetic step from docosahexaenoic acid ethyl esters (Et-DHA) due to the direct H/D-exchange with deuterium oxide (D₂O) catalyzed by the complex [CpRu(CH₃CN)_{3]} PF₆ as shown in Scheme 1.

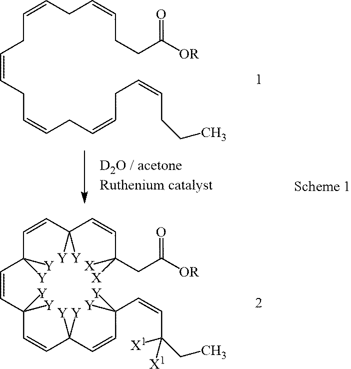

where R, X, X¹, and Y are as defined above.

As to Scheme 1, the reaction can be conducted using docosahexaenoic acid ethyl ester (or any other suitable ester), compound 1, a stoichiometric excess of deuterium oxide in a suitable inert solvent in the presence of the ruthenium catalyst as described in U.S. Pat. No. 10,577,304 which is incorporated herein by reference in its entirety.

As described herein, the synthetic methods employed limit the formation of the thermodynamic product produced. In Scheme 1, this is achieved by adjusting one or more of the reaction conditions. In one embodiment, the reaction time is reduced. In one embodiment, the reaction temperature is reduced. In one embodiment, the amount of catalyst employed is limited. Preferably, combinations of two or three of these embodiments are combined to minimize the amount of thermodynamic product formed.

In general, the amount of deuterium oxide employed is generally from about 150 to 200 equivalents per equivalent of compound 1. The deuterium oxide is added to an inert solvent such as acetone. The amount of catalyst employed is generally from about 1 to about 2.5 weight percent based on the amount of compound 1 used. The inert solvent is used in sufficient quantities to render the catalyst and deuterium oxide miscible in the resulting solution and to dissolve compound 1. The reaction is conducted at from about 15° to about 26° C., and preferably from about 19° to about 23° C., for a period of time sufficient to achieve sufficient deuteration of compound 1 while limiting the amount of the thermodynamic product formed. Typically this is about 5 to 7 hours and preferably 5 to 6 hours.

After reaction completion, the resulting mixture is first treated with benzene, toluene, and the like to kill the catalyst. The now destroyed catalyst is removed by charcoal, titanium dioxide, imidazole, carboxyimidazole, benzimidazole, 2-carboxy-benzimidazole, 4-carboxybenzimidazole, 5-carboxybenzimidazole, 6-carboxy-benzimidazole, thiazole, 2-carboxythiazole, 4-carboxythiazole, 5-carboxythiazole, cysteine, mercaptonicotinic acid, salicylic acid, 2-thiolbenzoic acid, 2-aminobenzoic acid, EDTA, combinations of two (or more) of the above, and the like. The solvent and deuterium oxide are stripped away under vacuum to provide the resulting product.

As shown in Example 1 and the Appendix attached (which is incorporated herein by reference in its entirety), the total amount of deuteration at the bis-allylic sites ranges from about 92 to about 97 percent. Stated differently, after deuteration the 10 hydrogen atoms at the bis-allylic sites have been replaced with, on average, between about 9.2 and about 9.7 deuterium atoms leaving only about 0.3 to 0.8 hydrogen atoms. Moreover, high field NMR establishes that on average from about 87 to about 92 percent of the carbon atoms at the bis-allylic sites have two deuterium atoms and from more than about 5 about to 12 percent of the carbon atoms at the bis-allyic sites have a single hydrogen and a single deuterium substitution with the residual being CH₂ moieties.

Given the above, it has been determined that even though complete deuteration of the bis-allylic sites has not been achieved, the presence of CHD groups at these sites impart greater stability against lipid peroxidation than the CH₂ groups. By limiting the reaction conditions such that, on average, no more than about 2 percent of the carbon atoms at the bis-allylic sites are CH₂ groups, the resulting composition still provides for excellent control of against LPO in vivo.

### Pathology

The resulting pathology of each of the oxidative retinal diseases is different from the underlying etiology of the disease. That is to say that whatever divergent conditions trigger each of these oxidative retinal diseases (the etiology), once triggered the pathology of these diseases involves the accumulation of oxidized DHA products. By limiting the oxidative damage, the pathology of the disease is addressed. In the case of AMD as an example, animal studies evidence that the degradation of eyesight is significantly limited by treating the animal with deuterated DHA as compared to the untreated animals.

Without being limited to any theory, the incorporation of deuterated DHA into the outer segments of rods and cones of the retina and surrounding retinal tissues limit the degree of oxidation by reactive oxygen species. This, in turn, protects the cells in the retina from damage and destruction typical of AMD.

### Methodology

In one embodiment, the methods described herein comprise the administration of deuterated DHA to a patient suffering from an oxidative retinal disease. The drug is delivered to the patient at a dose prescribed by attending clinician. Typically, such a dose is from about 0.1 to about 1.0 grams/day. The accumulation of deuterated DHA in the body can be monitored by, for example, blood tests to ensure that the patient is accumulating deuterated DHA consistent with achieving a therapeutic result. If the blood tests evidence insufficient levels of deuterated DHA, the clinician can determine if the dietary intake of DHA should be adjusted, dosing should be increased, or if a change from the loading dose to the maintenance dose might be delayed. Specific examples of methods for administering DHA are found in U.S. Provisional Pat. Applications Serial Nos. 63/224,674; 63/224,679; and 63/224,690 each of which is incorporated herein by reference in its entirety.

The methods described herein may include administration of deuterated DHA or an ester thereof to a patient in order to accumulate a therapeutic concentration of deuterated DHA for use in the methods described herein.

In one embodiment, deuterated DHA or ester thereof is administered to the patient in sufficient amounts to generate a concentration of deuterated DHA in a patient (e.g., in the red blood cells, plasma, and/or retinal cells) of at least about 50%, preferably at least about 60%, more preferably at least about 70%, and most preferably at least about 80%, based on the total amount of DHA, including deuterated DHA, found therein. In an embodiment, the percentage of deuterated DHA compared to total DHA in a patient (e.g., in the red blood cells, plasma, and/or retinal cells) may be between about 50% and about 80%, between about 50% and about 70%, or between about 50% and about 60%.

### Combinations

The therapy provided herein can be combined with any other treatments used with oxidative retinal diseases provided that such treatment does not interfere with the therapy described herein. In the case of macular degeneration, drugs such as bevacizumab, ranibizumab. aflibercept, and brolucizumab have all been prescribed to attenuate disease progression and can be used in combination with the therapy described herein.

In another embodiment, a combination therapy can employ a drug that operates via an orthogonal mechanism of action relative to the methods described herein. Suitable drugs for use in combination include, but not limited to, antioxidants such as edaravone, idebenone, mitoquinone, mitoquinol, vitamin C, or vitamin E, riluzole which preferentially blocks TTX-sensitive sodium channels, conventional pain relief mediations, and the like.

### Pharmaceutical Compositions

The specific dosing of deuterated DHA (drug) is accomplished by any number of the accepted modes of administration. As noted above, the actual amount of the drug used in a daily or periodic dose per the methods of this invention, i.e., the active ingredient, is described in detail above. The drug can be administered at least once a day, preferably once or twice or three times a day.

This invention is not limited to any particular composition or pharmaceutical carrier, as such may vary. In general, compounds of this invention will be administered as pharmaceutical compositions by any of a number of known routes of administration. However, orally delivery is preferred typically using tablets, pills, capsules, and the like. The particular form used for oral delivery is not critical.

Pharmaceutical dosage forms of a compound as disclosed herein may be manufactured by any of the methods well-known in the art, such as, by conventional mixing, tableting, encapsulating, and the like. The compositions as disclosed herein can include one or more physiologically acceptable inactive ingredients that facilitate processing of active molecules into preparations for pharmaceutical use.

The compositions can comprise the drug in combination with at least one pharmaceutically acceptable excipient. Acceptable excipients are non-toxic, aid administration, and do not adversely affect the therapeutic benefit of the claimed compounds. Such excipient may be any solid, liquid, or semi-solid that is generally available to one of skill in the art. One such excipient is a consumable oil such as oleic acid (e.g. olive oil), canola oil and other well known consumable oils. Such oils may also contain an emulsifier, a sweetner, a colorant, a preservative and other well known ancillary materials.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Other suitable pharmaceutical excipients and their formulations are described in Remington’s Pharmaceutical Sciences, edited by E. W. Martin (Mack Publishing Company, 18th ed., 1990).

The compositions as disclosed herein may, if desired, be presented in a pack or dispenser device each containing a daily or periodic unit dosage containing the drug in the required number of subunits. Such a pack or device may, for example, comprise metal or plastic foil, such as a blister pack, a vial, or any other type of containment. The pack or dispenser device may be accompanied by instructions for administration including, for example, instructions to take all of the subunits constituting the daily or periodic dose contained therein.

The amount of the drug in a formulation can vary depending on the number of subunits required for the daily or periodic dose of the drug. Typically, the formulation will contain, on a weight percent (wt %) basis, from about 10 to 100 weight percent of the drug based on the total formulation, with the balance, if any, being one or more suitable pharmaceutical excipients. Preferably, the compound is present at a level of about 50 to 99 weight percent.

In preferred embodiment, the drug is encapsulated inside a capsule without the need for any pharmaceutical excipients such as stabilizers, antioxidants, colorants, etc. This minimizes the number of capsules required per day by maximizing the volume of drug in each capsule.

### Testing Protocols

Once administered, the attending clinician needs to monitor the rate of absorption of the deuterated DHA into the retina. As physical access to the retina is not feasible, the methods described in the examples illustrate that either blood plasma or red blood cells (RBCs) can be used as a proxy for assessing whether absorption is proceeding properly. This is because the plasma and RBCs both reach steady state concentrations which occur at different times after the start of therapy nevertheless allow for the clinician to determine if the patient has reached steady state concentrations. When steady state concentrations are reached, then the clinician is assured that the maximum concentration deuterated DHA is found in the blood which feeds the retina and, thereof, the retina is receiving the appropriate amounts of deuterated DHA.

Testing blood plasma or RBCs for individual components contained therein is well established in the art. The examples below evidence one method for making such assessments. However, the testing protocol used is not critical as long as the test analysis provides for a ratio of deuterated DHA in the blood or plasma based on the total amount of DHA present in the sample - including both non-deuterated DHA and deuterated DHA.

However, as each patient has a different rate of absorption of deuterated PUFA in general and deuterated DHA in particular, based on diet and their unique physiology as evidenced in the Examples, testing the patient for appropriated uptake of deuterated DHA is necessary. In addition, such testing can assess whether the patient is being compliant with dosing instructions provided by the attending clinician. In one embodiment, the method includes restricting the patient’s consumption of dietary DHA to no more than 132 mg/day during therapy with deuterated DHA. In one embodiment, the method includes restricting the patient’s consumption of dietary DHA to no more than 71 mg/day during therapy with deuterated DHA. In one embodiment, the method includes restricting the patient’s consumption of dietary DHA to no more than 62 mg/day during therapy with deuterated DHA. In one embodiment, the method includes restricting the patient’s consumption of dietary DHA to no more than 54 mg/day during therapy with deuterated DHA. In one embodiment, the consumption of dietary DHA is restricted to no more than about 54 mg to about 132 mg per day, no more than about 62 mg to about 132 mg per day, or no more than about 71 mg to about 132 mg per day.

The method of testing is not critical as long as the test analysis provides for a ratio of deuterated DHA in the blood or plasma based on the total amount of DHA present in the sample - including both non-deuterated DHA and deuterated DHA.

In addition to the above, standardize curves to establish timing and concentrations to reach steady state in blood for different dosing regimens for deuterated DHA can be generated for each dose of DHA. As shown in the Examples, standardized curves for determining the time between initiation of therapy and when steady state concentrations are provided for two different markers - blood plasma and RBCs using two different dosing regimens. Using such procedures, a standardized curve for any dosing concentration of deuterated DHA can be established.

### EXAMPLES

This invention is further understood by reference to the following examples, which are intended to be purely exemplary of this invention. This invention is not limited in scope by the exemplified embodiments, which are intended as illustrations of single aspects of this invention only. Any methods that are functionally equivalent are within the scope of this invention. Various modifications of this invention in addition to those described herein will become apparent to those skilled in the art from the foregoing description and accompanying figures. Such modifications fall within the scope of the appended claims.

As used herein, the following abbreviations have the following definitions. Terms that are not defined have their accepted scientific definitions.

### Example 1: Synthesis of D-PUFAs

200 grams of docosahexaenoic acid ethyl ester, 1.9 kg of D20 (about 170 equivalents), about 3.8 to about 4 grams of [CpRu (CH₃CN)_{3]} PF₆, are combined with acetone in an amount sufficient to homogenize the reaction mixture (the total volume of the reaction mixture is about 9 liters).

The isotope exchange process is maintained for about 5-7 hours at a temperature of from about 19° to about 23° C. Afterwards, the reaction mixture is treated to kill the catalyst and the solution is worked up. The recovered product can be assessed by conventional techniques including HPLC, NMR, MS and the like.

The ¹H NMR peak for hydrogen atoms at the bis-allylic site are at 2.8 ppm downfield from TMS. In the absence of catalytic deuteration, integration of this peak corresponds to ten (10) protons as the natural abundance of deuterium oxide is negligible. After catalytic deuteration, integration of this peak and correlation to the proton peak for starting DHA allows for estimation of the extent of deuteration at the bis-allylic sites.

The ¹H NMR spectrum can also be used to estimate the degree of deuteration from two mono-allylic positions, and to obtain data for each of these positions, since they give signals at different values of the chemical shift due to the proximity or distance from the carboxylate ester.

### Example 2: Determination of the D-DHA Substitution Rates in Plasma, Red Blood Cells and Retina

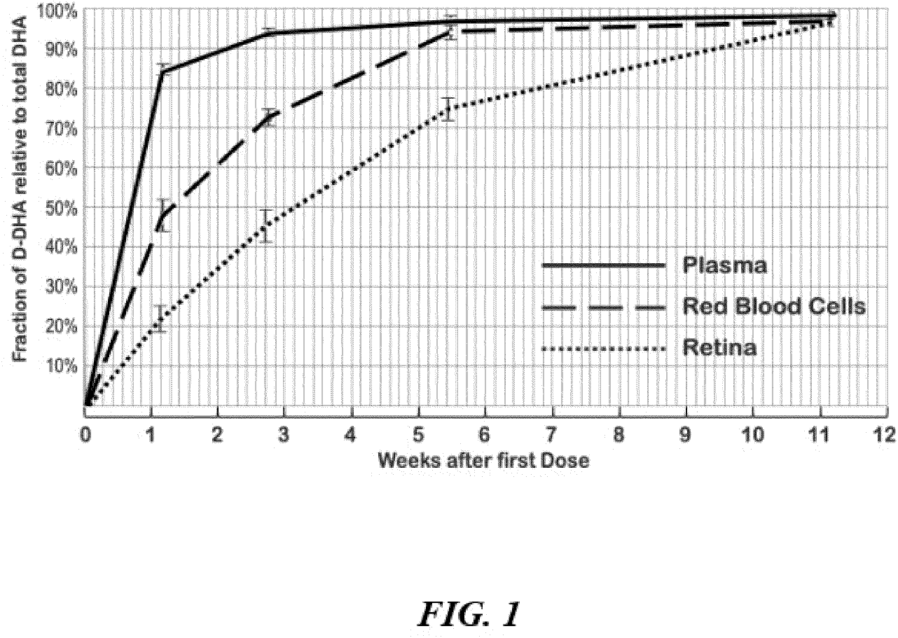

Mature adult C57BL/6J mice were fed for 78 days with a customized rodent diet containing 0.5% w/w D-DHA and no natural DHA. The animals were sacrificed, and plasma, red blood cells and retinal tissues were dissected at study Days 8, 19, 38, 78, (six animals per time point, 3 males + 3 females). D-DHA + DHA were extracted from samples and derivatized into methyl esters with a mixture of heptane/ toluene (63:37 by volume), and methanol/ dimethoxypropane/ sulfuric acid (85:11:4 by volume) by gentle shaking at 80° C. for 2 hours, followed by separation and drying down of the organic phase under nitrogen. D-DHA + DHA methyl esters were structurally identified and quantified by gas chromatography coupled with a tandem mass spectrometry detector and D-DHA substitution levels (in percent of total DHA) were calculated. The results are represented in Table 1 and graphed in FIG. 1. The measured data revealed that the D-DHA substitution rates follow a first order kinetics, i.e. they double at regular intervals in each sample type (e.g, about every 6-7 days in plasma, about every 10 days in red blood cells, and about every 20-22 days in retina whereby the maximum concentration (steady-state) is reached earlier in plasma and red blood cells (which are accessible in human subjects by a simple blood draw) than in retina (which is not accessible in living human subjects). With known doubling times, the steady-state concentrations can be calculated by single and/or multipoint measurements without the need to wait until steady-state is actually reached.

TABLE 1

D-DHA accretion in murine tissues D-DHA substitution levels (in percent of total DHA) at multiple time points after first exposure

| | | | | | |
|---|---|---|---|---|---|
| Sampling (Days) | 0 | 8 | 19 | 38 | 78 |
| Plasma | 0 | 84.78% | 94.17% | 97.34% | 98.43% |
| Red Blood Cells | 0 | 48.24% | 72.84% | 94.12% | 96.65% |
| Neuroretina | 0 | 20.89% | 42.94% | 70.74% | 92.10% |

### Example 3: Predicting Retinal D-DHA Substitution Rates From Plasma and Red Blood Cell Substitution Rates

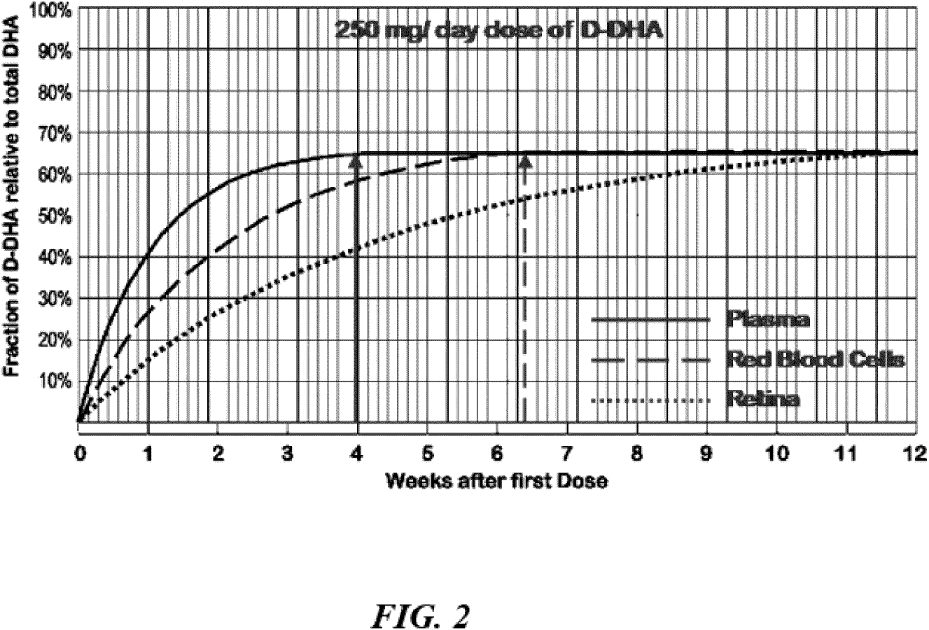

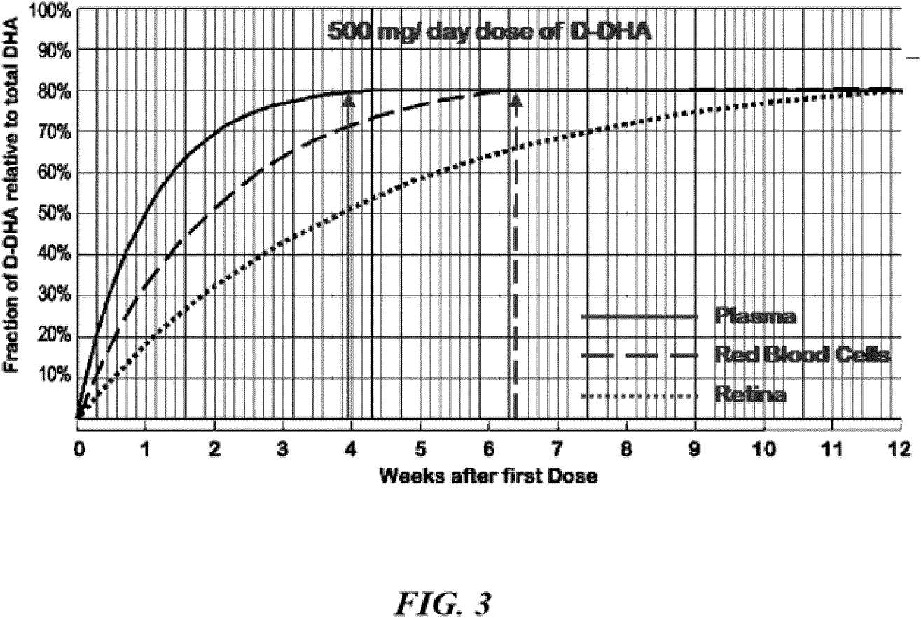

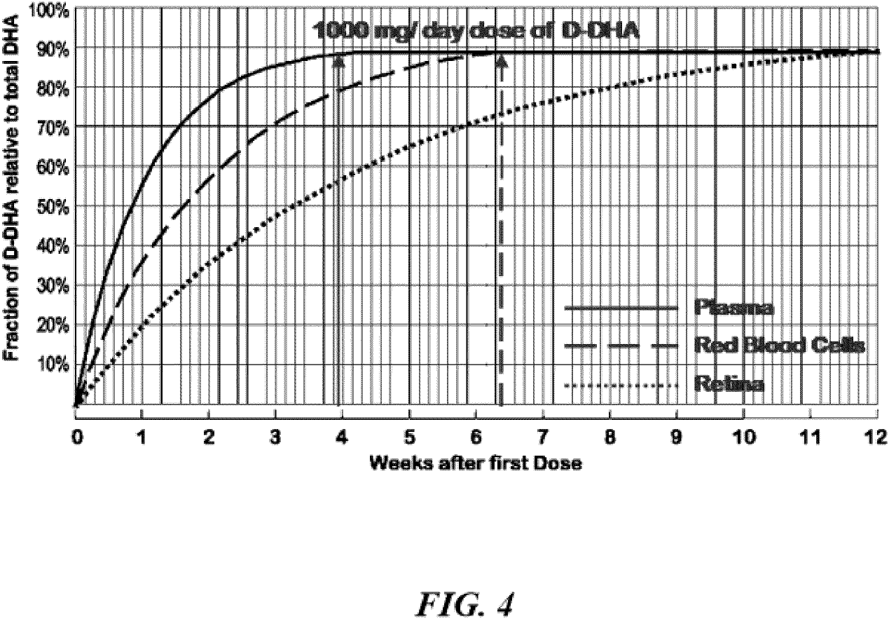

In contrast to controlled experimental diets, naturally occurring DHA is consumed by patients treated with D-DHA which can dilute the relative percentage of administered D-DHA absorbed with the total DHA pool and its final concentration at steady state in blood and eventually in the target tissue such as retina. The data and standard curves from Example 1 allow for the determination of the retinal steady-state D-DHA concentrations with reasonable accuracy ahead of time by calculating the D-DHA/ total DHA ratio in plasma and/or red blood cells. Table 2 exemplifies this using a mean daily dietary intake of about 130 mg of DHA per day (which represents the 90^{th} percentile of the mean usual DHA intake by males >51 years of age in the U.S.) FIGS. 2-4 illustrate how measuring plasma and red blood cell D-DHA concentrations can be used to predict expected retinal steady-state concentrations. With a known fixed daily dose and the measured D-DHA substitution rate at steady-state, the mean dietary DHA intake of individual patients can be approximated and monitored and permits either a timely adjustment of the daily D-DHA dose or dietary interventions to reduce natural DHA intake until the desired therapeutic D-DHA substitution levels, preferably about 50% or more, can be reached.

TABLE 2

Example for D-DHA steady-state substitution rates at 3 different fixed daily D-DHA doses at a given dietary background intake of natural DHA

| Fixed daily D-DHA dose | 250 mg | 500 mg | 1000 mg |
|---|---|---|---|
| DHA in human diet (example: 130 mg/day) | 130 mg | 130 mg | 130 mg |
| Total daily DHA intake (D-DHA + DHA) | 380 mg | 630 mg | 1130 mg |
| D-DHA substitution rate at steady-state (% of total DHA) | 66% | 79% | 88% |

### Example 4: Deuterated Decosahexanoic Acids Protects Against Oxidative Stress and Geographic Atrophy-Like Retinal Degeneration in a Mouse Model with Iron Overload

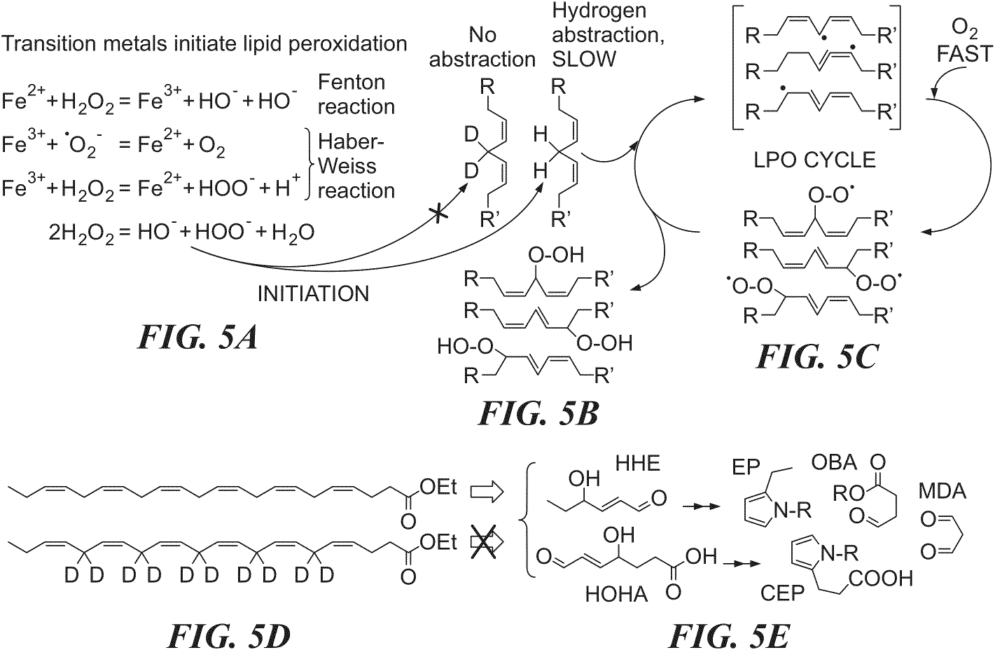

Oxidative stress plays a major role in the pathogenesis of neurodegenerative and retinal diseases. The retina is particularly susceptible to oxidative damage due to its high content of polyunsaturated fatty acids (PUFAs), photooxidation, high oxygen tension supplied from the choriocapillaris, and abundant mitochondria. PUFAs are essential constituents of cellular and mitochondrial membranes, and vital for optimal metabolism. PUFAs are vulnerable to oxidative stress, reacting with reactive oxygen species (ROS) through a lipid peroxidation (LPO) chain reaction. However, antioxidant therapies are unable to prevent LPO or neutralize secondary products of LPOs for stoichiometric reasons. Moreover, fully eradicating ROS may be detrimental because ROS can also modulate cell signaling. The abstraction of bis-allylic hydrogens is a rate-limiting step of ROS-driven PUFA oxidation. Substitution of deuterium atoms for hydrogen atoms at bis-allylic sites can slowdown the LPO chain reaction due to an isotope effect (FIGS. 5). PUFAs are unable to be synthesized in the human body de novo from carbon sources, e.g. acetate. Typically linoleic acid and alpha-linolenic acid, respectively, serve in the diet as the major precursors for biosynthesis of all the n-6 and n-3 PUFAs . D-PUFAs can be incorporated into mitochondrial and cellular membranes after oral dosing, replacing a fraction of the PUFAs naturally occurring in membranes, and conferring resistance to oxidative stress and LPO. D-PUFAs have been studied in multiple conditions involving oxidative stress and LPO. A deuterated version of linoleic acid (11,11-D₂-Lin; RT001) inhibited LPO and rescued cell death in both animal models and clinical trials in several neurodegenerative diseases, including Friedreich’s ataxia (FRDA), infantile neuroaxonal dystrophy (INAD), and progressive supranuclear palsy (PSP). D-PUFAs also reduced LPO and hold therapeutic potential in preclinical studies for Alzheimer’s, Parkinson’s, and Huntington’s diseases. Oxidative stress has been implicated in several retinal diseases, including age-related macular degeneration (AMD), light-induced damage, iron-related retinal degeneration, Leber’s hereditary optic neuropathy and retinitis pigmentosa.” Docosahexaenoic acid (cervonic acid; DHA, C22:6, n-3) is the most abundant PUFA in the retina, representing up to 40% of all total fatty acids in human rod photoreceptor outer segments. DHA is crucial for the integrity of photoreceptors and visual function. While ingestion of DHA-rich fatty fish is associated with lower AMD risk, n-3 PUFA supplementation has shown no appreciable benefits in patients with AMD or retinitis pigmentosa. Moreover, high doses of DHA may increase risk in conditions involving oxidative stress, due to its high sensitivity to oxidation.

The addition of DHA to the human RPE cell line ARPE-19 increased oxidative stress and LPO under high-intensity light exposure. Levels of carboxyethyl pyrrole (CEP), an immunogenic protein adduct specifically derived from the oxidation of DHA, are elevated in retinal tissues and plasma from patients with AMD. Further, immunization of mice with CEP adducts led to an AMD-like retinal degeneration. These pieces of evidence suggest that nonenzymatic oxidation of DHA in the retina may play a crucial role in the pathogeneses of retinal disorders involving oxidative stress.

In the present example, the impact is studied of deuterated DHA against oxidative stress and LPO in mice with iron-induced oxidative stress in the retina. A previously analyzed mouse model given intravitreal (IVT) injection of iron was found increased oxidative stress and CEP in the retina, followed by retinal pathologies similar to human AMD, including geographic atrophy of the RPE. Here, a mice was fed a diet containing an envelope of D-DHA isotopologues, with most prevalent being 6,6,9,9,12,12,15,15,18,18-D10-(4Z,7Z,10Z,13Z,16Z, 19Z)-docosa 4,7,10,13,16, 19-hexaenoic acid ethyl ester for 11 weeks followed by a wash-out in a pharmacokinetics study to establish a dosing regimen for efficient retinal uptake. To determine the protective effect of D-DHA against LPO, mice were fed a diet containing D-DHA for 1-4 weeks before giving an IVT injection of iron, or control saline. In mice fed with D-DHA for 4 weeks, >50% substitution of DHA with D-DHA in the neural retina was observed.

This regimen provided nearly complete protection against iron-induced retinal damage by inhibiting oxidative stress and DHA oxidation.

### Dietary D-DHA Efficiently Incorporated Into the Neural Retina and RPE Cells

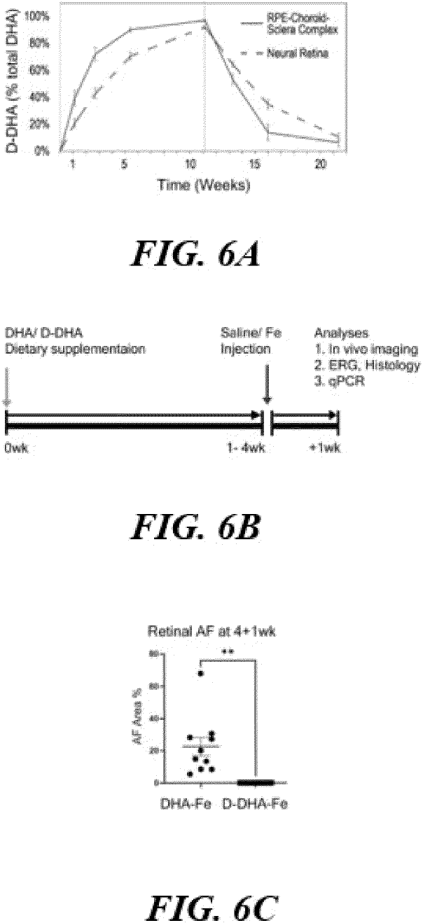

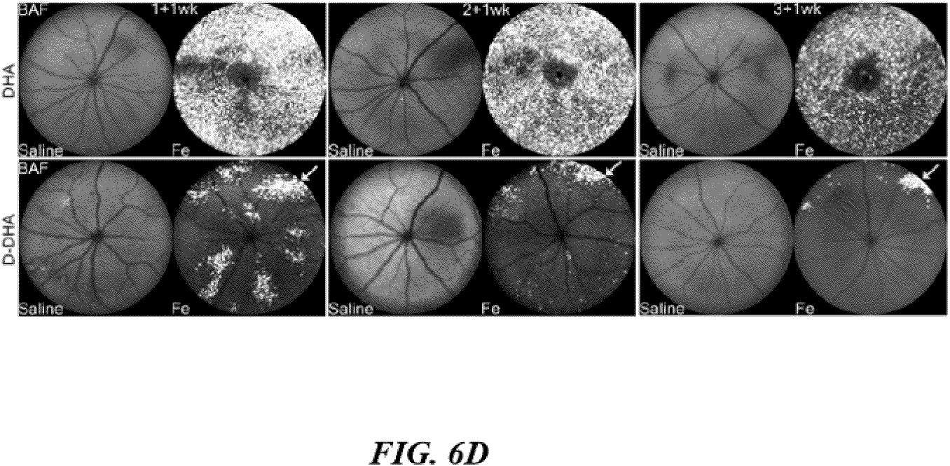

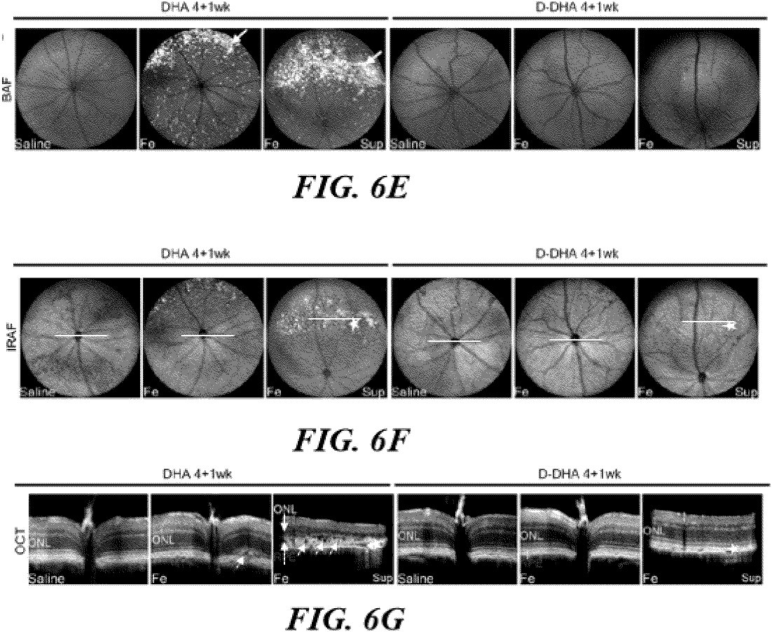

To determine the pharmacokinetics of ocular D-DHA uptake, incorporation and elimination from the neural retina and RPE/choroid/sclera, twelve-week-old C57BL/6J mice were fed a 0.5% D-DHA containing diet (0.5 g D-DHA/100 g food, Table 3) for 77 days followed by an additional 73-day wash-out phase with DHA. At 4 weeks, >55% of the DHA in the retina was D-DHA rising to >60% at 5 weeks (FIG. 6A). At similar time points, D-DHA in the RPE/choroid/sclera was >80%. Washout in the RPE/choroid/sclera was similarly more rapid than retina. Uptake and elimination followed classic first order kinetics. Based on the accretion and elimination data, two-month-old mice were fed with diets containing either D-DHA or natural DHA control for 1 week, 2 weeks, 3 weeks, and 4 weeks before the IVT injection of iron and saline control (FIG. 6B). In order to better approximate typical DHA doses in human prescription omega-3 supplements (e.g. 1,500 mg/day DHA in Lovaza), the D-DHA and DHA experimental mouse diets were adjusted to 0.25% instead of 0.5% for most of the study. On a 0.25% D-DHA diet, retinal D-DHA levels exceeded 50% at five weeks (52.2±1.5% and 55% of total DHA by our GC- and LC- based MS methods, respectively), regardless of whether eyes were injected with iron or control saline (Table 3). Table 3. shows D-DHA content as a percentage of D-DHA + DHA in neural retina and RPE from mice fed with D-DHA or DHA at 4 weeks, given IVT injections, then continued on the D-DHA diet for another week.).

TABLE 3

| Treatment | Tissue | % D-substitution |
|---|---|---|
| D-DHA + Fe | RPE cells | 59.3 ± 3.9% |
| D-DHA + saline | RPE cells | 60.8 ± 2.1 % |
| D-DHA + Fe | neural retina | 55.0 ± 3.3% |
| D-DHA + saline | neural retina | 55.0 ± 1.8% |

### D-DHA Protected Against Iron-Induced Retinal Autofluorescence (AF) and Degeneration

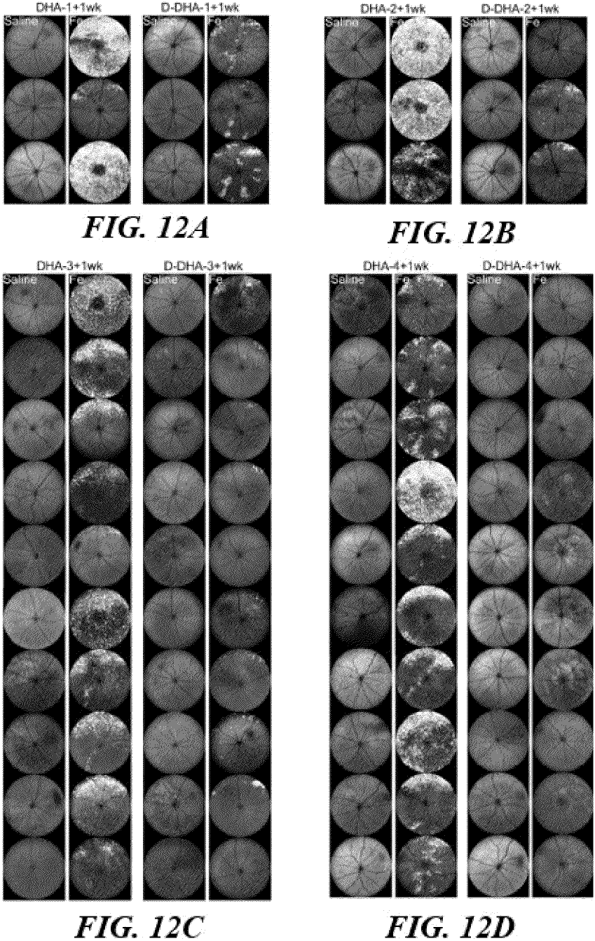

In a prior study, IVT iron was shown to induce retinal AF and degeneration. To evaluate the protective effect of 0.25% D-DHA diet, confocal scanning laser ophthalmoscopy (cSLO) and optical coherence tomography (OCT) were employed for in vivo imaging at 1 week after injections. For the cSLO imaging, both blue autofluorescence (BAF) and near-infra autofluorescence (IRAF) were performed. At 1 week after iron injection, BAF images of mice fed with natural DHA displayed intense hyper-autofluorescent spots, representing photoreceptor layer undulations, as well as autofluorescent RPE and myeloid cells (FIGS. 6D and 6E). These same retinas imaged with IRAF showed hyper and hypo autofluorescence in the superior retina. BAF and IRAF images of mice fed with D-DHA revealed a dose-dependent reduction of iron induced retinal AF in mice fed with D-DHA for 1 week, 2 weeks, 3 weeks, and 4 weeks before the iron injection (FIGS. 6D and 6E, FIGS. 12). Optical coherence tomography (OCT) scans of mice fed with natural DHA showed marked thinning of the outer nuclear layer in the superior retina at 1 week after iron injection (FIG. 6G). In contrast, mice fed with D-DHA for 4 weeks showed complete protection of retinal structure in OCT scans (FIG. 6G).

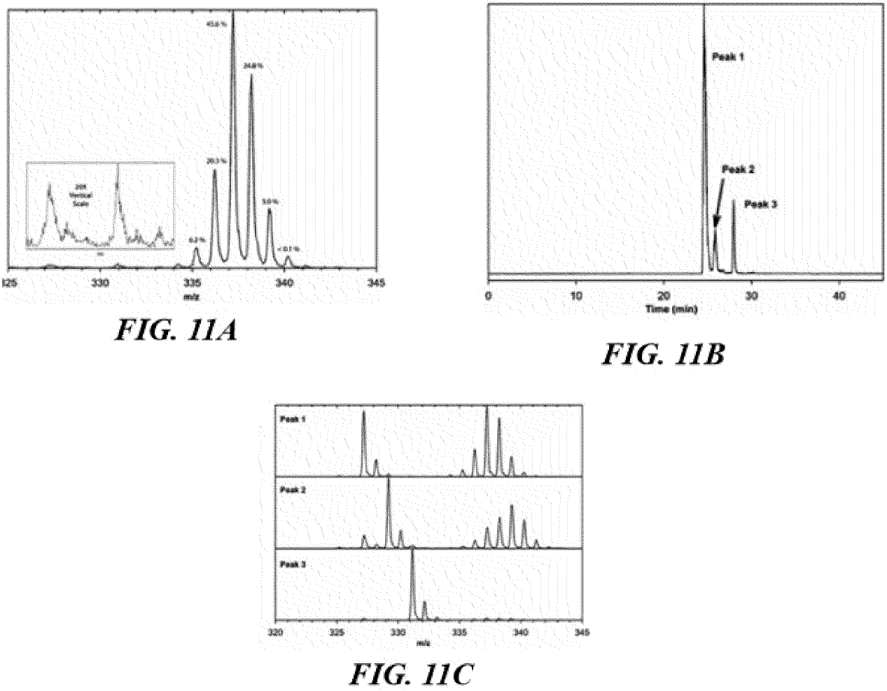

The extent to which D-DHA replaced natural DHA was determined by LC/MS by feeding an experimental diet containing 0.25% D-DHA for increasing periods of time. Microdissected samples of neural retina and RPE from mice fed the experimental diet for 4 weeks were analyzed after given IVT iron or control saline, and then continued on the experimental diet for another week. It was confirmed that 22.6% of DHA in the control diet consisted of isotopomers due to natural abundance ¹³C and no D-DHA. Therefore, signals from the 327.2/283.2 transition represent 78.4% of the total DHA. The experimental diet contained no detectable natural DHA, but rather a distribution of deuterium-substituted DHA isotopologues (FIGS. 11A-C), which is a consequence of the D-DHA preparation method. After corrections were applied for natural abundance ¹³C, it was determined that the D₁₀-DHA isotopologue comprised 45.6% of the D-DHA species in the analyzed sample, with Ds-DHA, D₉-DHA, D₁₁-DHA, and D₁₂-DHA isotopologues comprising the balance. Therefore, signals from the 337.2/293.2 transition represented 45.6% of the D-DHA. With these corrections applied, D-DHA isotopologues as a percentage of total DHA (i.e., D-DHA + DHA) was determined to be 59.3-60.8 % in isolated RPE, and 55.0 % in neural retina. There were no significant differences between iron-treated and saline-control eyes (Table 3).

### D-DHA Protected Retinal Function and Histologic Structure

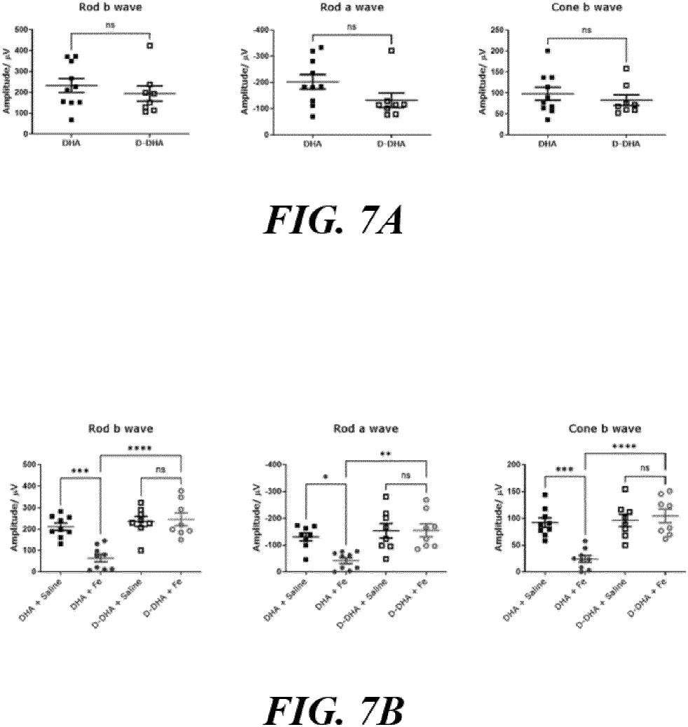

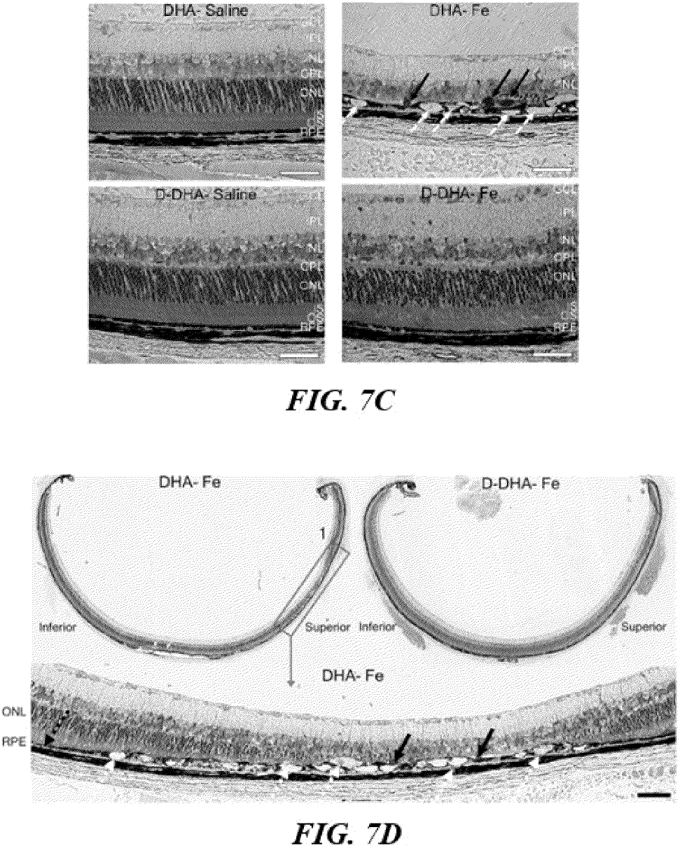

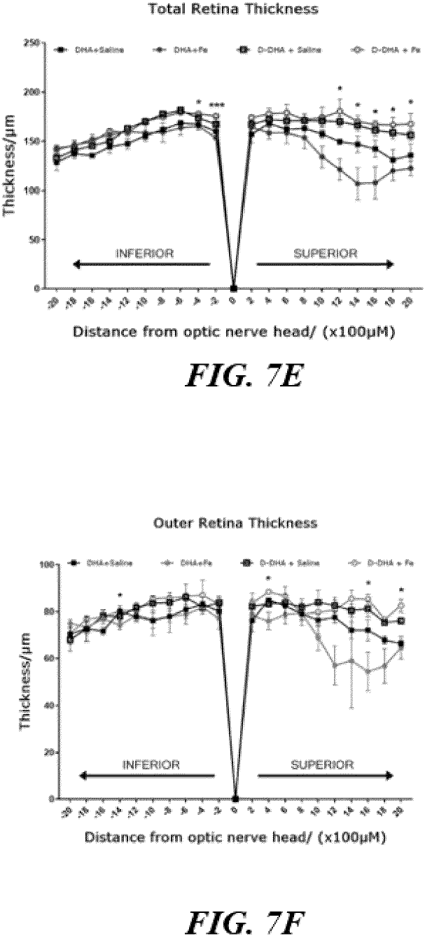

Electroretinography was conducted on mice fed with D-DHA or natural DHA for 4 weeks to evaluate retinal function. In mice not given IVT injections, dosing with D-DHA caused no significant difference in the rod-b wave, rod-a wave and cone-b wave amplitudes compared to those fed with natural DHA. Thus, using this measure, incorporation of D-DHA had no impact on retinal function (FIG. 7A). In mice fed with control DHA, 1 week after iron injection, the rod b-wave, rod a-wave and cone-b wave amplitudes were significantly decreased in the eyes injected with iron compared with saline control, consistent with iron-induced retinal damage. Iron injected eyes from mice with ≥50% retinal D-DHA had marked protection of rod b-wave, rod a-wave and cone-b wave amplitudes compared to the DHA plus iron injection group (FIG. 7B). There was no significant difference between the saline-injected eyes and the iron-injected eyes from mice on the D-DHA diet, indicating complete anatomical and functional protection. Toluidine blue staining was performed on plastic sections to examine retinal histology. At 1 week after iron injection, the outer nuclear layer (ONL) was thinned in the superior retinas of mice fed with DHA, with intracellular vesicles in degenerated RPE cells (white dashed arrows), and myeloid cells infiltrating between the neural retina and RPE layer (black solid arrows) (FIGS. 7C and 7D). Mice fed with D-DHA showed complete protection of retinal structure against the toxicity of iron injection (FIGS. 7C and 7D). Quantification of total retina thickness and outer retina thickness in IVT iron injected eyes from DHA-fed mice displayed a reduction in the superior retina. In contrast, IVT iron injected eyes from D-DHA fed mice were significantly protected and not different from saline injected eyes (FIGS. 7E and 7F). Taken together, >50% retinal D-DHA substitution led to complete protection of retinal function and structure against iron induced damage.

### D-DHA Prevented the Formation of CEP, a Unique Oxidation Product of DHA

Iron-catalyzed peroxidation of phospholipids containing DHA leads to unique carboxyethyl pyrrole (CEP) adducts not formed from any other PUFA. CEP has been detected by IHC in human AMD eyes and mouse retinas, including those from mice receiving IVT iron. To test whether D-DHA could protect against iron-induced CEP formation, mice were fed with D-DHA or DHA for 4 weeks prior to IVT injection of iron or control saline.

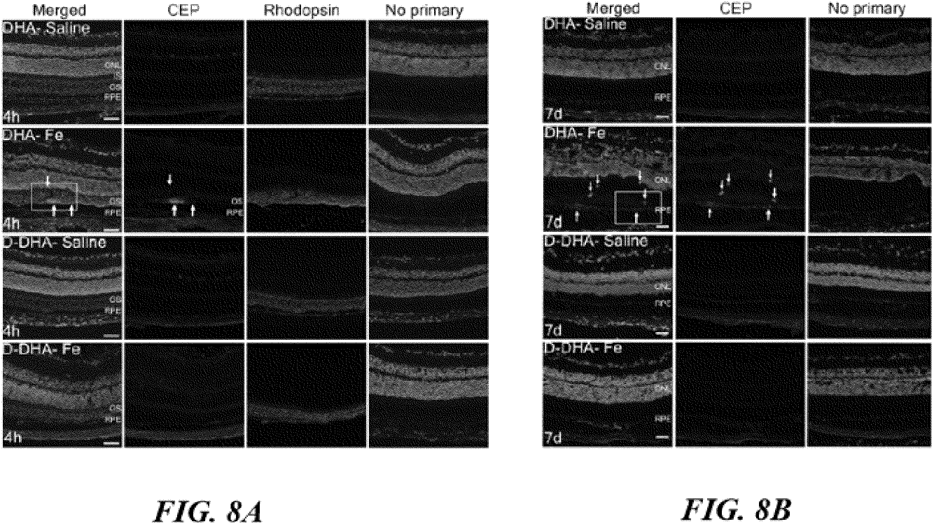

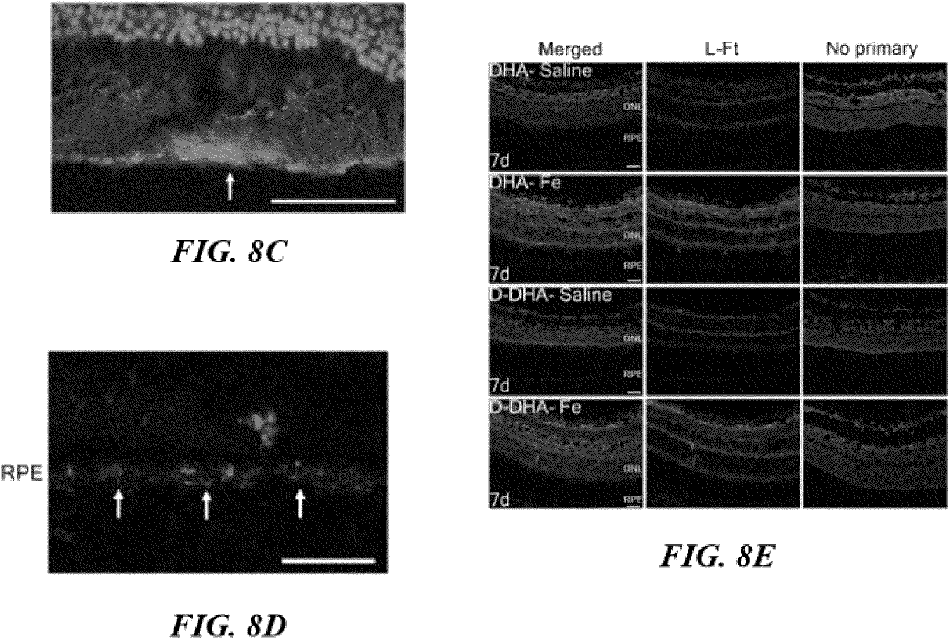

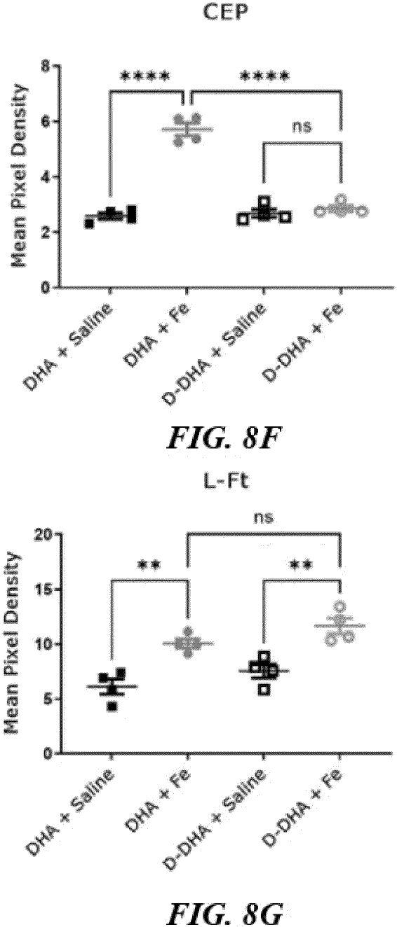

Cryosections were prepared at 4h and 1 week after injections. Co-labelling for CEP and rhodopsin was conducted to assess and localize CEP. At 4 hours after injection, increased immunolabelling for CEP was present in rhodopsin co-labeled photoreceptor outer segments in IVT iron injected eyes of DHA fed mice but not in IVT iron injected eyes of D-DHA fed mice (FIG. 8A). At 1 week after injection, immunolabelling for CEP localized to RPE and infiltrating myeloid cells in IVT iron injected eyes of DHA fed mice, likely originationg from phagocytosed oxidized photoreceptor outer segments (FIG. 8B). Immunolabelling for CEP was undetected in D-DHA fed mice. These results indicate that iron induced the accumulation of CEP, and D-DHA at >50% retinal substitution prevented the accumulation of CEP by inhibiting DHA oxidation. Immunolabeling for ferritin light chain (L-Ft) was conducted to assess retinal iron levels and localization, since L-Ft protein levels are increased in response to elevated intracellular iron. At 1 week after saline injection, L-Ft weakly labeled the ganglion cell layer, outer plexiform layer, and inner segment layers (FIG. 8E). Increased L-Ft staining was observed in the inner plexiform layer, outer plexiform layer, and inner segments in both the DHA/IVT iron and D-DHA/IVT iron mice (FIG. 8E). These two groups were not different from each other, indicating that D-DHA did not prevent IVT iron-induced iron accumulation in retinal cells; instead, D-DHA blocked its downstream toxic effects. Quantification of pixel density for CEP and L-Ft label was conducted using ImageJ software (FIGS. 8F and 8G), and quantitatively verified the results described above.

### D-DHA Protected Against mRNA Changes Indicative of Iron-Induced Retinal Cell Death, Oxidative Stress, and Inflammation

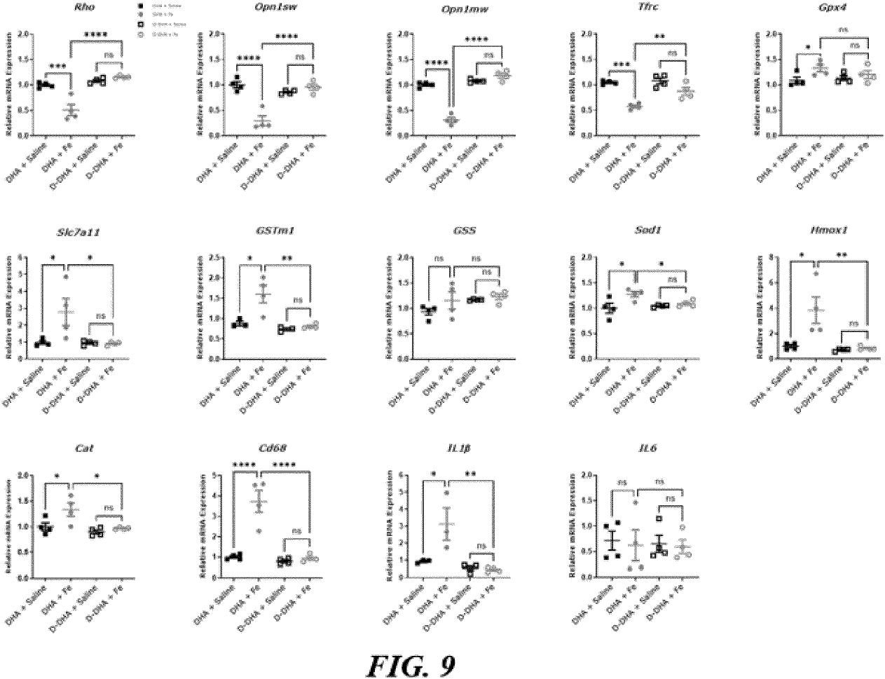

Quantitative PCR was used to evaluate mRNA changes in the neural retinas of mice fed with D-DHA or DHA for 4 weeks. Cell-type specific, iron regulating, antioxidant, and inflammation related genes were evaluated at 1 week after iron or saline injections. The mRNA levels of the rod-specific gene rhodopsin (Rho), cone-specific gene cone opsin1 medium wave sensitive and short wave sensitive (Opn1mw and Opn1sw) were measured to assess the stress and differentiation of rod and cone photoreceptors. The mRNA levels of Rho, Opn1sw, and were significantly decreased in the neural retinas of mice fed with DHA that received IVT iron, compared to IVT saline controls. In contrast, there was no change in these mRNAs in the neural retinas of D-DHA/IVT iron mice relative to IVT saline controls (FIG. 9). The mRNA levels of transferrin receptor (Tfrc) which are inversely related to intracellular iron levels, can be used as an indicator of intracellular iron levels. At 1 week after iron injection, Tfrc mRNA levels in the neural retina were significantly decreased in DHA/IVT iron and D-DHA/IVT iron mice indicating iron loading in the neural retinas of both groups. These two groups had slightly different Tfrc levels, perhaps as a result of loss of some photoreceptors in the DHA/IVT iron group (FIG. 9). The mRNA levels of antioxidants solute carrier family 7 member 11 (SLC7A11), glutathione peroxidase 4 (GPX4), glutathione S-transferase isoform m1(GSTm1), glutathione synthesis (GSS), catalase (Cat), heme oxygenase 1 (Hmox1), and superoxide dismutase 1 (Sod1) were measured to investigate oxidative stress. The mRNA levels of Slc7a11, Gpx4, GSTm1, Cat, Hmox1, and Sod1 were significantly increased in the neural retina of DHA/IVT iron compared with saline injected eyes. This upregulation of antioxidants was prevented in D-DHA/IVT iron eyes, with no significant difference between iron and saline injected eyes in mice fed with D-DHA. The mRNA levels of IL1β, IL6 and cluster of differentiation 68 (Cd68) were detected to investigate the retinal inflammation. The mRNA levels of IL1β and Cd68 were significantly increased in DHA/IVT iron retinas compared to saline controls, but were not increased in the D-DHA/IVT iron retinas. The mRNA levels of Glutathione-synthase (GSS) and IL6 were not increased by IVT iron in mice on either diet (FIG. 9). Taken together, 250% retinal D-DHA can significantly protect against iron induced oxidative stress, photoreceptor cell damage, and inflammation in the neural retina.

### D-DHA Prevented Iron-Induced Geographic Atrophy Development

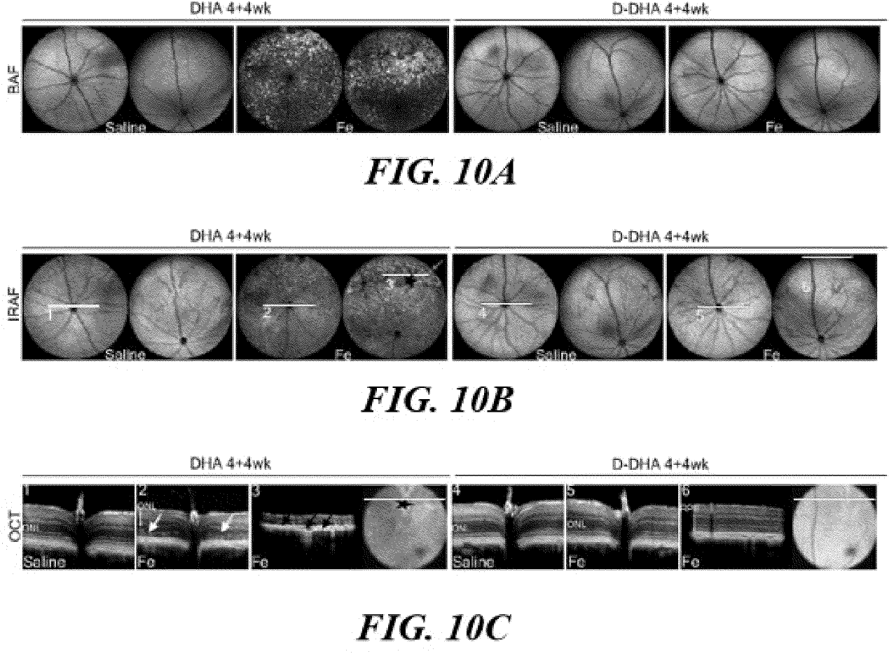

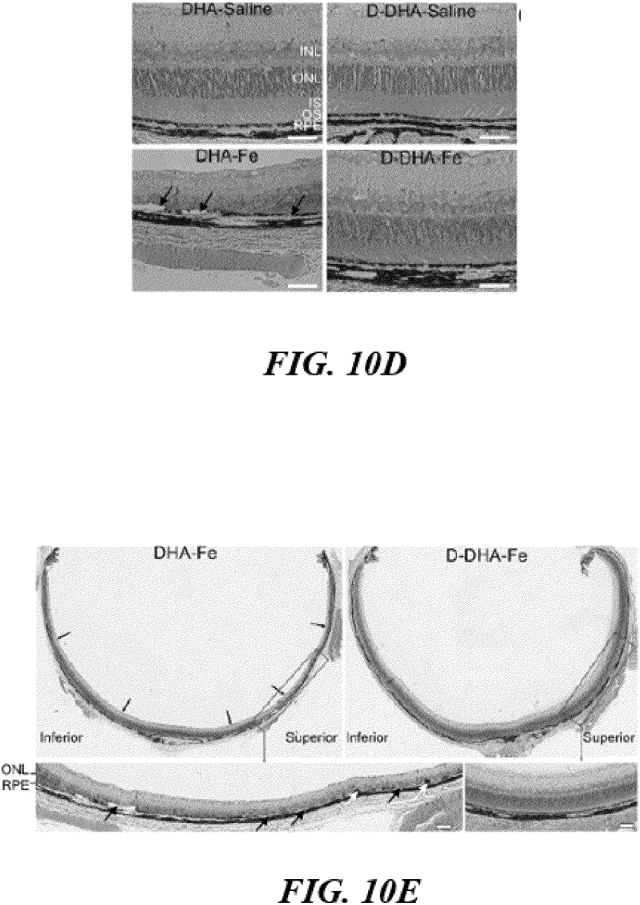

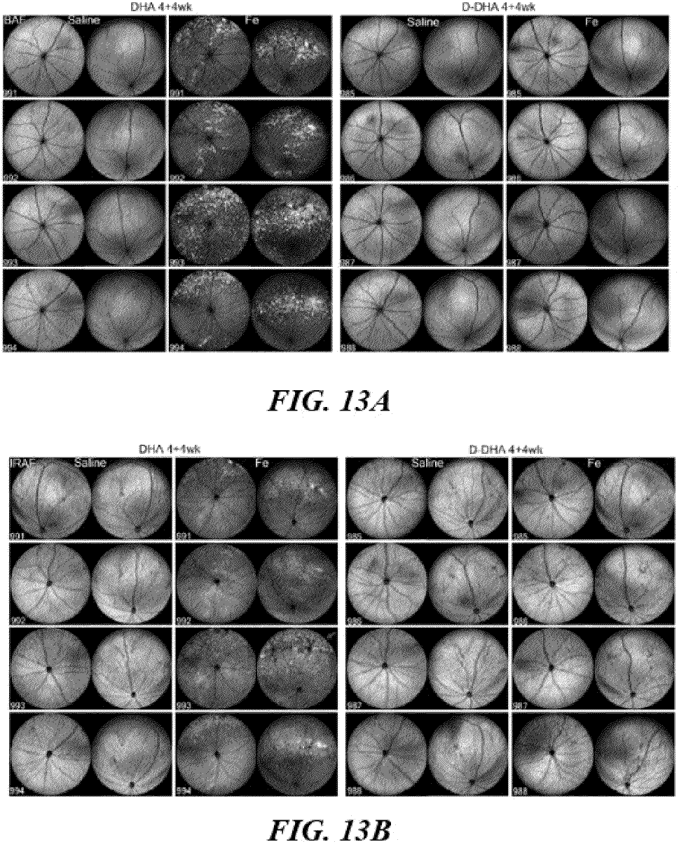

Mice given D-DHA for 4 weeks showed complete retinal protection 1wk after iron injection (FIGS. 6). Our previous study showed geographic atrophy in the superior retina within a month of IVT iron injection. To evaluate whether D-DHA could protect against geographic atrophy in this model, mice were continued on respective diets for 4 weeks after iron or saline injection. At this point, BAF and IRAF images displayed hypo-AF in the superior retinas of mice fed with DHA, similar to geography atrophy (FIGS. 10A and 10B). This corresponded to photoreceptor (white arrows) and RPE degeneration (black arrows) in OCT scans (FIG. 10C). D-DHA fully protected against the geographic atrophy development (FIGS. 10A-C). cSLO and OCT scans were obtained from multiple mice, and all displayed the protective effect of D-DHA on chronic retinal degeneration (FIGS. 13A-B). Taken together, 250% retinal D-DHA provided long-term protection against the geographic atrophy development induced by iron.

### Discussion

In this example, analysis was performed on whether the inhibition of DHA oxidation might prevent oxidative stress and retinal degeneration in a mouse model with retinal iron overload. IVT iron was observed to induce retinal AF, oxidative stress, accumulation of carboxyethyl pyrrole (CEP), a DHA-specific oxidation product, and photoreceptor degeneration followed by progressive geographic atrophy, replicating features of human AMD. In this example, it was demonstrated that dosing of D-DHA completely protected against all these iron-induced retinal changes.

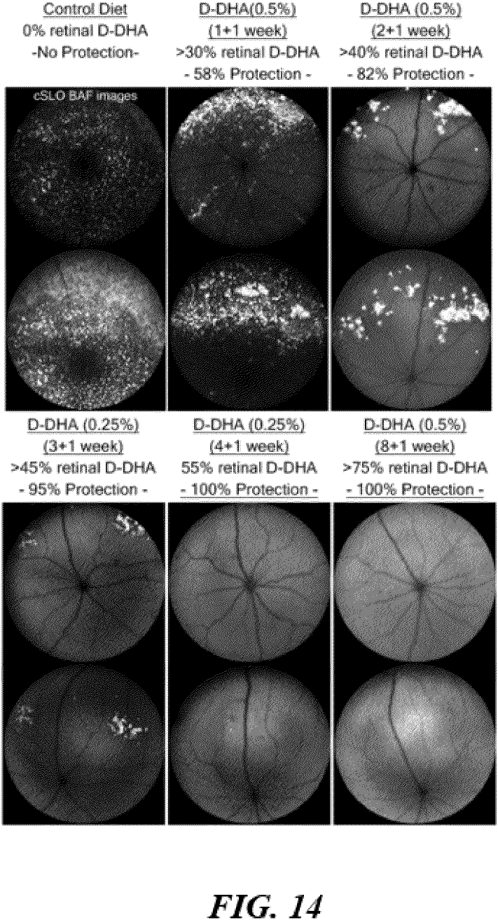

Mice fed with D-DHA for 1 week, 2 week, and 3 weeks prior to the iron injection showed a dose-dependent reduction in iron induced retinal AF and retinal degeneration with >50% protective effects already observed at >30% retinal D-DHA substitution levels (FIG. 14). After D-DHA reached 50% retinal substitution levels in mice fed with D-DHA for 4 weeks prior to the iron injection, complete protection of retinal structure and function was observed.

D-DHA inhibited oxidative stress and LPO, which is particularly pernicious because of its autocatalytic radical chain reaction cycle and nonenzymatic nature. Hydrogen abstraction at the bis-allylic sites is the rate limiting step of LPO. PUFAs deuterated at the bis-allylic positions inhibit this step due to the isotope effect. D-DHA prevented oxidative stress-induced increases in mRNA levels of antioxidants GSTm1, Catalase, Sod1, Hmox1, Gpx4, and Slc7all. In addition, immunolabelling of CEP was undetected in the retina of mice with ≥50% retinal D-DHA substitution levels prior to iron injection, suggesting the deuteration can inhibit the oxidation of DHA and the accumulation of its toxic derivative CEP, contributing to retinal protection. CEP (FIG. 5E) is a DHA-specific, adduct-forming oxidation product. CEP adducts have been found increased in drusen deposits and plasma of AMD patients, and elevated in the retinas of rodents after intense light exposure. Mice immunized with CEP adducts accumulated complement component-3 in Bruch’s membrane, drusen deposits underneath the RPE, and RPE degeneration, features of dry AMD. CEP adducts also stimulated neovascularization in vivo through a VEGF-independent pathway.

LPO has been shown to be detrimental to cells in multiple ways. For example, it can make lipid bilayers leaky and stiff. On a chemical level, LPO can generate small molecule species such as lipid hydroperoxides, prostaglandin-like isoprostanes, and isoketals which have primarily detrimental effects. Another group of LPO products has been implicated in numerous pathologies comprises activated carbonyls, including malondialdehyde, 4-HNE (from n-6 PUFA; fat-soluble) and 4-HHE (from n-3 PUFA; aqueous-soluble). These are highly reactive (FIG. 5E) and can irreversibly cross-link phospholipids, proteins, and cause DNA transversions. By virtue of inhibiting LPO, D-PUFAs reduce the levels of these compounds Moreover, D-PUFAs can cross-protect various H-PUFAs; a membrane-incorporated D-PUFA protects other PUFAs in this membrane by terminating the LPO chain reaction. For example, the presence of the n-6 PUFA D2-linoleic acid in lipid bilayers downregulated not just 4-HNE but also 4-HHE formation.

A threshold protective effect has been shown for various D-PUFAs on the stability of liposomes under oxidative stress, revealing a strong protective effect of D-DHA, which efficiently inhibited the LPO even when present at 1% fraction of the total PUFAs in a lipid membrane in vitro. In this example, mice with ≥50% D-DHA incorporation in the neural retina and RPE cells showed a complete protection effect against the oxidative damage induced by iron. This is probably because of the high overall content of DHA in the retina and up to 30 mol% of rod outer segment membrane phospholipids carrying twin DHA acyl chains (supraenoic phospholipids with more than six double bonds). This unique feature of photoreceptor outer segments may require ≥50% D-DHA levels to completely insulate proximal unprotected supraenoic DHA chains from each other. The high levels of hydroxyl radicals generated by Fe through the Fenton reaction and the subsequent LPO cycle in this drastic model may demand high concentrations of retinal D-DHA while lower levels might be sufficient in less severe oxidative conditions. D-DHA inhibited the oxidative damage associated inflammation in the neural retina. At 1 week after injection, the mRNA levels of IL1β and Cd68 had no significant difference between iron and saline injected neural retina from mice fed with D-DHA, which were significantly increased in the iron injected neural retina from mice fed with DHA. Oxidative stress and lipid peroxides can induce the inflammatory response, including the infiltration and activation of microglia and macrophages, and the secretion of pro-inflammatory cytokines such as IL1β, IL-6, TNF-α and others. Overall, the results suggest that D-DHA can prevent the neuro-inflammation induced by iron. D-DHA showed long term protection against geographic atrophy. Mice with ≥50% retinal D-DHA levels were completely protected against the chronic development of geographic atrophy in the superior retina, compared to mice fed with DHA. Moreover, it was found that IVT iron induced damage appears to be less severe with the DHA diet than in a previous study in mice fed with “regular chow” without DHA (LabDiet 5001). It was previously observed that iron-induced retinal AF throughout the retina in mice fed with LabDiet 5001 occurred at 1 week after iron injection followed by “kidney bean” shaped geographic atrophy that typically occurred at 4 weeks after iron injection. In the current example, we observed that iron induced AF was more limited to the superior retina in mice fed with DHA for 4 weeks at 1 week after iron injection (FIGS. 12), and the full “kidney bean” shaped geographic atrophy only occurred in some, but not all mice (FIGS. 13), which was correlated with the amount of superior region AF one week after iron injection. Additional studies can be performed to determine the basis of the more severe iron induced retinal degeneration in mice on LabDiet 5001, which differs from the DHA control diet used in this study. Since the only difference between the control DHA diet and D-DHA diet used herein is whether DHA is deuterated or not deuterated, these results suggest that D-DHA leads to a long-term protection against photoreceptor and RPE degeneration induced by iron overload. D-PUFAs have been reported to inhibit LPO in several mouse models of neurological diseases associated with oxidative stress, including Parkinson’s disease, Alzheimer’s disease, Huntington’s disease, Infantile neuroaxonal dystrophy. RT001 (D2-Lin ethyl ester) has been tested in clinical trials of Friedreich’s ataxia ⁸ and infantile neuroaxonal dystrophy showing notable safety and tolerability. Here, we report the protective effect of D-DHA in retinal disease in vivo, using a mouse model replicating features of human AMD. The results indicate that D-DHA can prevent iron induced retinal degeneration by inhibiting oxidation of DHA. D-DHA may be a viable therapeutic for retinal pathogenesis involving oxidative stress and lipid peroxidation.

### Materials and Methods

### D-DHA Synthesis

D-DHA was synthesized as previously described (e.g., AV Smarun, M Petkovic, MS Shchepinov, D Vidović. Site-Specific Deuteration of Polyunsaturated Alkenes. The Journal of organic chemistry. Dec. 15, 2017;82(24):13115-13120) Catalytic exchange results in an assortment of D-DHA isotopologues from D6-D12, centered at D10 which is typically 30-40% of the total bis allylic isotopologues. At least 90% of D-DHA is reinforced with two Ds at all bis allylic carbons, and the remaining 10% are reinforced with at least one D at each of the bis allylic positions.

### Ocular D-DHA Accretion and Elimination

Eleven-week-old C57BL/6J mice were purchased from Jackson Laboratory (Bar Harbor, ME) and housed in 20-25 lux light conditions under 12 h day/night cycle in the Dean McGee Eye Institute Animal Research Facility at the University of Oklahoma Health Sciences Center, Oklahoma City, OK. One week after acclimatization to the vivarium with ad libitum access to laboratory rodent chow and water the animals were assigned to experimental groups. To determine ocular D-DHA accretion, the mice were switched from the laboratory rodent diet to the experimental D-DHA supplemented diet containing 0.5% D-DHA plus 6.5% high oleic soybean oil (w/w) in AIN93G⁵¹ (Research Diet, Inc. New Brunswick, NJ). The diets were vacuum packaged and stored at - 20° C. Food was replaced three times a week with fresh food ad libitum that is stored in 4° C. after it is taken from - 20° C. Based on previous estimated retina accretion kinetics, retina, optic nerve and eye cups containing sclera, retinal pigment epithelium-choroid (RPE-choroid) were dissected from 6 mice (3 females and 3 males) at different time points during eleven weeks of D-DHA feeding. The tissues were snap frozen in liquid nitrogen, and stored in -80° C. until fatty acid analyses. After eleven weeks (77 days) on D-DHA diet, the animals were switched to the washout diet containing 0.5% DHA plus 6.5% high oleic soybean oil (w/w) in AIN93G and maintained on this diet until euthanasia at three different time points up to 73 days, when tissues were harvested for fatty acid analysis. All animal procedures were approved by the University of Oklahoma Health Sciences Center Institutional Animal Care and Use Committee.

### Iron Induced Acute RPE Atrophy

Adult male wild-type C57BL/6J mice (Stock No.000664, Jackson Labs, Bar Harbor, ME, USA) were housed in standard conditions under cyclic light (12 h:12 h light-dark cycle). Mice had ad libitum access to water and food. Beginning at 2 mo of age, mice were placed on the AIN93G diet described above, supplemented with 0.25% D-DHA or DHA (control diet) for 1 week, 2 weeks, 3 weeks, or 4 weeks prior to the intravitreal injection. The complete composition of the of DHA and D-DHA containing diets is shown in Table 4. Mice were given an intravitreal injection of 1 µl 0.5 mM ferric ammonium citrate diluted in 0.9% NaCl (saline) (MP Biomedicals LLC, Santa Ana, CA) or

1 µl of saline as control. Intravitreal injections were performed as previously described ⁵⁴. Mice were continued on respective diet until their final evaluation. All housing and procedures were performed according to the NIH Guide for the Care and Use of Experimental Animals and approved by the University of Pennsylvania Animal Care and Use Committee.

TABLE 4

| | DHA Diet | | D-DHA Diet | |
|---|---|---|---|---|
| Product # | D20120104 | | D20120105 | |
| | gm% | kcal | gm% | kcal |
| Protein | 14 | 13 | 14 | 13 |
| Carbohydrate | 67 | 63 | 67 | 63 |
| Fat | 11.3 | 24 | 11.3 | 24 |
| Total | | 100 | | 100 |
| kcal/gm | 4.2 | | 4.2 | |
| Ingredient | gm | kcal | gm | kcal |
| Casein | 140 | 560 | 140 | 560 |
| L-Cystine | 1.8 | 7.2 | 1.8 | 7.2 |
| Corn Starch | 454.5 | 1818 | 454.5 | 1818 |
| Maltodexbin 10 | 125 | 500 | 125 | 500 |
| Sucrose | 100 | 400 | 100 | 400 |
| Cellulose, BW200 | 50 | 0 | 50 | 0 |
| Coconut Oil, 101 (Hydrogenated) | 80.3 | 723 | 80.3 | 723 |
| D6-Arachidonic Acid Ethyl Ester (ARA-Et-D) | 0 | 0 | 0 | 0 |
| D-DHA, EE (Docosah ‘c Acid Ethyl Ester; DHA-Et-D) | 0 | 0 | 2.6 | 23 |
| D-Linoleic Acid, ethyl ester modified | 0 | 0 | 0 | 0 |
| H-Arachidonic Acid, Ethyl Ester (ARA-Et-H) | 0 | 0 | 0 | 0 |
| H-DHA, EE (Docosahexaenoic Acid Ethyl Ester; DHA-Et-H) | 2.6 | 23 | 0 | 0 |
| H-Linoleic Acid, Ethyl Ester (Ethyl Linoleate) | 0 | 0 | 0 | 0 |
| H-Linolenic Acid, Ethyl Ester (Ethyl Linolenate) | 207 | 19 | 2.07 | 19 |
| Oleate, Ethyl | 0 | 0 | 0 | 0 |
| Sunflower Oil, High Oleic (typical 83.6% Oleate per :nig) | 31.7 | 285 | 31.7 | 285 |
| t-Butylhydroquinone | 0.008 | 0 | 0.008 | 0 |
| Mineral Mix S10022M | 35 | 0 | 35 | 0 |
| Vitamin Mix V10037 | 10 | 40 | 10 | 40 |
| Chdine Bitartrate | 2.5 | 0 | 2.5 | 0 |
| Cholesterol | 0 | 0 | 0 | 0 |
| FD&C Yellow Dye #5 | 0.04 | 0 | 0 | 0 |
| FD&C Red Dye #40 | 0.01 | 0 | 0.025 | 0 |
| FD&C Blue Dye #1 | 0 | 0 | 0.025 | 0 |
| | | 4375 | | 4375 |
| Total | 1035.528 | | 1035.528 | |
| Coconut Oil, Hydrogenated (gm/100 gm diet) | 7.75 | | 7.75 | |
| 06-Machidonic Acid Ethyl Ester (ARA-Et-D; gm/100 gm diet) | 0 | | 0 | |
| D-DHA, EE (D10-Docosahexaenoic Acid Ethyl Ester, DHA-Et-D, gLn/100 gm diet) | 0 | | 0.25 | |
| H-Arachkionic Acid, Ethyl Ester (ARA-Et-H; gm/100 gm diet) | 0 | | 0 | |
| H-DHA EE (Docosahexaenom Acid Ethyl Ester DHA-Et-H, gm/100 gm diet) | 0.25 | | 0 | |
| H-Linoleic Acid, EE (Ethyl Linoleate, gm/100 gm diet) | 0 | | 0 | |
| H-Linolenic Acid, EE (Ethyl Linolenate, gm/100 gm diet) | 0.2 | | 0.2 | |
| Sunflower Oil, High Oleic (gm/100 gm diet) | 3.1 | | 3.1 | |

### Mass-Spectrometry

Three types of mass spectrometric analysis were performed to confirm repeatability of results. In the first, lipids were extracted from retinas by a modified Folch method (CHCl₃/CH₃OH, 2:1). derivatized to fatty acid methyl esters (FAME), and analyzed by high resolution capillary gas chromatography and specialized chemical ionization tandem mass spectrometry. Baseline resolved DHA and D-DHA total ion signal were integrated, and the proportions of D-DHA/total DHA were calculated. The second type of mass spectrometric analysis was performed on samples of the control and experimental diets, as well as microdissected neural retina and RPE from animals on the experimental diet. Lipids were extracted and saponified, and analyzed by ESI-LC/MS on a 4000 QTrap (Sciex) operating in enhanced negative mode over an m/z range of 320 - 345 and a scan rate of 250 /sec. This analysis verified that the control diet contained DHA but no detectable D-DHA, while the experimental diet contained an array of D-DHA isotopologues but only trace amounts of natural DHA (FIGS. 11). Peaks corresponding to DHA with 8, 9, 10, 11, 12, and 13 deuterium substitutions were readily identified in the experimental diet, and in samples of neural retina and RPE. The relative distribution of DHA isotopologues in neural retina and RPE samples was indistinguishable from the relative distribution in the experimental D-DHA supplemented diet.

The third type of mass spectrometric analysis was performed on microdissected neural retina and RPE from animals on the two diets using the same extraction and chromatographic procedures. However, ESI-LC/MS analysis was performed in negative multiple reaction monitoring mode for transitions 327.2/283.2 (corresponding to 78.4% of ordinary DHA) and 337.2/293.2 (corresponding to 45.6% of the deuterium containing DHA). Results for D-DHA are reported as a percentage of total DHA (i.e. D- DHA + DHA).

### In Vivo Imaging System

Mice were given general anesthesia and placed on a platform. Pupils were dilated with 1% tropicamide saline solution (Akorn, Inc., Lake Forest, IL). Optical coherence tomography (OCT) imaging was performed for visualization of the retinal structure by using a Bioptigen

Envisu (R2200, Bioptigen Inc., Durham, NC, USA) coupled to a broadband LED light source (T870-HP, Superlum Diodes, Ltd, Ireland). Confocal scanning laser ophthalmoscopy (cSLO) (Spectralis HRA, Heidelberg Engineering, Franklin, MA, USA) was used for visualization of retinal AF using BluePeak™ or simply blue AF (488 nm excitation) and near-infrared AF (787 nm excitation) imaging modes.

### Electroretinography

Mice were dark adapted overnight and anesthetized with the same procedure. The electroretinograms were recorded with an Espion E3 system (Diagnosys LLC, Lowell, MA) with a ganzfeld Color Dome stimulator as previously described. All electroretinography were performed at the same time of day.

### Tissue Preparation and Immunofluorescence

Immunofluorescence on cryosections was conducted as described previously. Primary antibodies used: mouse anti CEP (1:200, a kind gift of John Crabb, Cleveland Clinic, OH); rabbit anti-rhodopsin (1:200; Abcam); rabbit anti L-FT (1:1000, a kind gift of Maura Poli and Paolo Arosio, University of Brescia, Italy). Images were acquired with an epifluorescence microscope (Nikon 80i microscope, Nikon, Tokyo, Japan), and analyzed using NIS-Elements (Nikon).

### Plastic Sections and Toluidine Blue Staining

Plastic sections (3 µm) were cut in the sagittal plane. The third eyelid was used for the orientation when embedding eye cups. Toluidine blue staining on plastic sections was used to evaluate retinal morphology.

### RNA Extraction and Quantitative RT-PCR

Neural retina tissues were isolated as previously described (e.g., M Hadziahmetovic et al. Age-dependent retinal iron accumulation and degeneration in hepcidin knockout mice. Investigative ophthalmology & visual science. Jan. 5, 2011;52(1):109-18).Gene expression changes in the neural retina and purified RPE cells were evaluated. Gapdh was used as an endogenous control. Taqman Probes (ABI, Grand Island, NY, USA) were used as follows: Rho (Mm00520345), Opn1mw (Mm00433560), Opn1sw (Mm00432058), Tfrc (Mm00441941), Slc7all (Mm00442530), Gpx4 (Mm00515041), GSS (Mm00515065), GSTm1 (Mm00833915), Cat (Mm00437992), Sod1 (Mm01700393), Hmox1 (Mm00516005), Cd68 (Mm03047343), IL- 1β (Mm00434228), IL-6 (Mm00446190). The amount of target mRNA was compared among the groups of interest. All reactions were performed in technical (3 reactions per eye) triplicates and biological replicates (3-5 mice per genotype).

### Statistical Analysis

Statistical analyses were performed using GraphPad Prism 6.0 (San Diego, CA). One-way analysis of variance (ANOVA) was performed, and post-hoc analysis was employed using Tukey-Kramer testing when differences were observed in ANOVA testing (p <0.05). Mean ± SEM was calculated for each group.

What is claimed is:

1. A method for monitoring a patient for uptake of deuterated docosahexaenoic acid wherein said method comprises:
periodically administering to said patient an effective dose of deuterated docosahexaenoic acid or an ester thereof;
obtaining one or more blood samples from said patient after the start of therapy;
assessing the amount of deuterated docosahexaenoic acid in said sample relative to the total amount of docosahexaenoic acid;
comparing the assessed amount of deuterated docosahexaenoic acid against a standard concentration curve wherein said curve is based on a specific dose of deuterated docosahexaenoic acid or ester thereof employed, the blood component being assessed, and the said length of time from start of therapy; and
determining if the patient is properly absorbing deuterated docosahexaenoic acid based on said curve.

2. The method of claim 1, wherein the blood component being assessed is plasma.

3. The method of claim 1, wherein the blood component being assessed is red blood cells.

4. The method of claim 1, wherein the length of time between start of therapy and testing is from about 3 to about 45 days.

5. The method of claim 4, wherein the length of time between start of therapy and testing is at least about 14 days.

6. The method of claim 4, wherein the length of time between start of therapy and testing is at least about 30 days.

7. A method for monitoring a patient for uptake of deuterated docosahexaenoic acid wherein said method comprises:
periodically administering to said patient an effective dose of deuterated docosahexaenoic acid or an ester thereof wherein said does is about 250 mg/day;
obtaining one or more plasma samples from said patient after the start of therapy;
assessing the amount of deuterated docosahexaenoic acid in said sample relative to the total amount of docosahexaenoic acid;
comparing the assessed amount of deuterated docosahexaenoic acid against a standard concentration curve wherein said curve is based on the said length of time from start of therapy; and
determining if the patient is properly absorbing deuterated docosahexaenoic acid based on said curve.

8. A method for monitoring a patient for uptake of deuterated docosahexaenoic acid wherein said method comprises:
periodically administering to said patient an effective dose of deuterated docosahexaenoic acid or an ester thereof wherein said does is about 500 mg/day;
obtaining one or more red blood cell samples from said patient after the start of therapy;
assessing the amount of deuterated docosahexaenoic acid in said sample relative to the total amount of docosahexaenoic acid;
comparing the assessed amount of deuterated docosahexaenoic acid against a standard concentration curve wherein said curve is based on the said length of time from start of therapy; and
determining if the patient is properly absorbing deuterated docosahexaenoic acid based on said curve.

9. A method for monitoring a patient for uptake of deuterated docosahexaenoic acid wherein said method comprises:
periodically administering to said patient an effective dose of deuterated docosahexaenoic acid or an ester thereof wherein said does is about 1,000 mg/day;
obtaining one or more red blood cell samples from said patient after the start of therapy;
assessing the amount of deuterated docosahexaenoic acid in said sample relative to the total amount of docosahexaenoic acid;
comparing the assessed amount of deuterated docosahexaenoic acid against a standard concentration curve wherein said curve is based on the said length of time from start of therapy; and
determining if the patient is properly absorbing deuterated docosahexaenoic acid based on said curve.

10. The method of claim 1, wherein the concentration of deuterated docosahexaenoic acid is less than that provided by the standardized curve, then the clinician has the option of either modifying the patient’s diet to reduce the amount of DHA-containing fat consumed per day and/or to increase the amount of drug administered.
